(19) United States
(12) Patent Application Publication
(10) Pub. No.: US 2024/0285506 A1
(43) Pub. Date: Aug. 29, 2024

(54) ACRYLIC NAIL ENHANCEMENTS

(71) Applicant: Esschem, Inc., Linwood, PA (US)

(72) Inventors: Shane Philippi, Woolwich Township, NJ (US); Patricia Ferry, Wilmington, DE (US); Easley Wallace, Jr., Berwyn, PA (US); Susan Sheariss, Swedesboro, NJ (US); Howard Slaff, Wilmington, DE (US)

(73) Assignee: Esschem, Inc., Linwood, PA (US)

(21) Appl. No.: 18/564,826

(22) PCT Filed: Jun. 24, 2022
(86) PCT No.: PCT/US2022/034909

§ 371 Date: Nov. 28, 2023

Related U.S. Application Data
(60) Provisional application No. 63/216,241, filed on Jun. 29, 2021.
(60) Provisional application No. 63/222,137, filed on Jul. 15, 2021.

(51) Int. Cl. A61K 8/81; A45D 31/00; A61K 8/41; A61Q 3/02

(52) U.S. Cl. CPC A61K 8/8147; A45D 31/00; A61K 8/411; A61Q 3/02; A45D 2200/25; A61K 2800/43; A61K 2800/95

(57) ABSTRACT

A composition is described for use in preparing human nail applications, the composition comprising: a polymer component formed from: a first (meth)acrylate ester with an alkyl side chain of 3 or less carbon atoms; a second (meth)acrylate ester with a primary, secondary or tertiary alkyl side chain of at least 5 carbon atoms; and an initiator; or alternatively, (i) a polymer component formed from: a first (meth)acrylate ester with an alkyl side chain of 3 or less carbon atoms; and a second (meth)acrylate ester with a primary, secondary or tertiary alkyl side chain of at least 5 carbon atoms; and an initiator, in combination with (ii) a monomer component comprising: a first (meth)acrylate ester, where the (meth)acrylate ester is di-, tri-, or multi-functional; a second (meth)acrylate ester with an alkyl side chain of 3 or less carbon atoms; and an accelerator. A kit containing the composition and a method for preparing the composition are also described.

### CROSS-REFERENCE TO RELATED APPLICATIONS

This application claims priority to U.S. Provisional Patent Application No. 63/216,241, filed on Jun. 29, 2021, titled “REDUCED ODOR ACRYLIC NAIL ENHANCEMENTS” and U.S. Provisional Patent Application No. 63/222,137, filed on Jul. 15, 2021, titled “REDUCED ODOR ACRYLIC NAIL ENHANCEMENTS” the entirety of each of which is incorporated by reference herein for all purposes.

### FIELD OF INVENTION

This invention relates to self-curing monomer/polymer compositions useful for forming artificial nails and protective coatings on human nails, where the compositions have reduced odor and/or a reduced number or size of entrained defects, exhibit superior handling characteristics and are essentially clear when applied on human nails to protect, adorn, extend and/or decorate them.

### BACKGROUND OF THE INVENTION

Artificial nails are popular items of personal fashion. Acrylic monomers and polymers can be combined to create artificial nails. An acrylic nail is traditionally prepared and applied in a salon by trained and licensed personnel. With increasing frequency, however, individual consumers are buying home use kits and applying the artificial nails themselves. These compositions may also be used to mend and/or strengthen natural and artificial nails. The ingredients, the application process and/or the finished result may be referred to as an acrylic nail enhancement system.

The art discusses a variety of self-curing compositions that can be applied to human nails for the purpose of forming an artificial nail. These preparations typically include two-part (meth) acrylic systems which consist of a liquid portion (herein referred to as a monomer or monomer liquid) and a powder portion (herein referred to as a polymer or polymer powder).

The term (meth)acrylate is recognized in the art as encompassing both acrylate and (meth)acrylate compounds. In the early history of the nail enhancement industry, methyl (meth)acrylate (MMA) was the primary monomer ingredient. In 1974, it was observed that MMA caused severe allergic reactions and was therefore replaced by ethyl (meth)acrylate (EMA).

The high vapor pressure of EMA undesirably results in an intense acrid smell during its use and curing. Conventional (as known in the marketplace, “commercial”, “conventional” or “established”) monomer liquids are comprised of primarily EMA. The odor is readily recognized by any retail customer entering a nail salon. Consumers tend to shun strong smelling substances on aesthetic grounds. The odor associated with EMA thus presents a use barrier for such monomer-polymer nail enhancement systems.

Chemical compounds used in nail salons have diverse smells, including those present in liquid nail polish (such as ethyl acetate, butyl acetate, ethyl alcohol, isopropyl alcohol), those in nail polish removers (such as acetone) and those in gel nail polish (such as various (meth)acrylic monomers and oligomers) as well as those in monomer-polymer nail enhancement systems (such as EMA). To address this issue, some salons seek to improve air circulation and ventilation, per National Institute for Occupational Safety & Health (NIOSH) recommendations. Alternatively, nail technicians use infection control masks to protect against inhalation of vapors, despite their lack of utility for that purpose. Other salons use a variety of scent diffusers, whether it be candles, bottles of fragrance or a more sophisticated distribution system. It would therefore be highly desirable for a monomer-polymer nail enhancement system to be less volatile and reduce unfavorable smells.

Low-odor (meth)acrylate nail liquids are well known and consist largely of slightly higher molecular weight monomers, compared to EMA. To resolve the acrid odor, a variety of approaches have been developed by the salon industry. Commonly known as “odorless monomers,” these liquid combinations produce less vapor during use than EMA.

There are any number of instrumental or mathematical analysis of odor. In the instance of an acrylic nail monomer, sensorial perception is the critical determination, for which, analysis by ASTM E-679-04 is industry standard. To assess relevance of the instant invention, “reduced odor”, “low-odor”, “lower odor” and/or “less odor” is a monomer liquid with an Odor Concentration less than 80% of commercial monomer (reference commercial monomer as shown in Example 1).

An early patent disclosure that addresses nail monomer odor is U.S. Pat. No. 4,871,534, where a variety of alkoxylated monomers are identified as the primary ingredients in the preferred monomer liquid, with secondary inclusion of functional (meth)acrylates as cross linkers. An analytical definition of “low-odor” is not specified. Primary monomer liquid ingredients are methoxyethoxyethyl (meth)acrylate and ethoxyethoxyethyl (meth)acrylate. Secondary ingredients include diethylene glycol di(meth)acrylate, 2-hydroxyethyl (meth)acrylate, trimethylolpropane tri(meth)acrylate and 1,12-dodecanediol di(meth)acrylate. All are used with a copolymer powder made of ethyl and methyl (meth)acrylate.

U.S. Pat. No. 5,098,696 identifies additional combinations of monomer liquid ingredients from those in U.S. Pat. No. 4,871,534, also with the claimed benefit of being odorless. An analytical definition of “low-odor” is not specified.

Reduced odor monomers, however, have disadvantages. While addressing the issue of undesired smells, use of such low-odor monomers remedies only one part of a two-part system. In particular, the incorporation of higher molecular weight and/or more functionalized acrylic compounds often complicates product application and/or finished enhancement quality. Reduced odor monomers are also typically poor solvents for use with the polymer powder and during use, often form an un-polymerized residue and/or thick oxygen inhibition layer, which must be removed. Use of reduced odor monomers can also require implementation of undesired changes to the application techniques that are preferred by nail technicians, and thus are resisted within the salon industry.

In addition, reduced odor monomers typically result in discolored nail enhancements. Common ingredients in such systems include alcohol derived and/or ethoxylated (meth)acrylates. The previous art uses various monomer compositions, such as ethylene glycol di(meth)acrylate (EGDMA), hydroxyethyl (meth)acrylate (HEMA), and/or methoxyethoxyethyl (meth)acrylate (diethylene glycol monomethyl ether (meth)acrylate).

Creating an artificial nail for application onto a human nail is an art and is often performed by trained and licensed personnel, but with increasing frequency also by the individual consumer. The following steps are a typical procedure for applying an artificial nail:
a) An artist's type brush is first dipped in the monomer liquid part. Doing so wets the brush fibers.
b) The wetted brush tip is then dipped into the polymer powder portion, allowing the monomer on the brush to absorb and begin to solvate the polymer powder. How effectively the wetted brush takes up the polymer powder is referred to as “pick up”.
c) The resulting heterogeneous mixture rapidly transitions from two phases of monomer and polymer-into one smooth and homogeneous mixture, e.g., a ball or slurry. How effectively the components transition into a smooth and homogenous phase is referred to as “wet out”.
d) This homogeneous mixture is then transferred, typically within 5 seconds, to the surface of a human nail. Deposition of the mixture, from the brush onto the nail, is referred to as “transfer”.
e) The transferred mixture is then shaped with the brush to form the desired coating and/or artificial nail extension. Ease of movement and supporting the intended form are referred to as “shaping”.
f) Upon application onto the nail, the viscosity of the monomer-polymer slurry increases to a point where it cannot reasonably be moved by the artist's brush.
g) Artistic forms may be attached to the end of the nail, to provide a surface on which to extend the artificial nail beyond the length of the natural nail.
h) Once applied and cured, the nail enhancement may be buffed with a manual or electric nail file, to shape the nail and smooth the surface as desired.

There is an industry expectation that once combined, the monomer and polymer components become homogeneous in no more than 10 seconds, preferably less. At that moment, the homogeneous mixture should have a viscosity of approximately 25,000 to 100,000 cPs. Conversely, reduced odor monomers cannot be readily picked up by (i.e., adsorbed and held onto) the nail technician's brush. Further, the liquid containing the reduced odor monomers is typically undesirably slow to wet out (i.e., combine with and begin the dissolution of) the polymer powder, which results in a delay or inability to form a homogeneous mixture. The viscosity of a typical reduced odor monomer liquid, approximately 30 seconds after combining with a conventional polymer powder, can be less than 10,000 cPs. This viscosity may represent an incomplete association of the liquid and powder system parts.

Preferentially, upon initiation by mixing, the viscosity of the monomer-polymer composition must increase, typically beyond 1,000,000 cPs. Otherwise, time is undesirably prolonged waiting for polymerization to occur, or if the applied monomer-polymer composition is not sufficiently viscous when applied to the nail, it will be difficult to shape and/or may even run off the nail surface. Once the monomer-polymer composition is applied to the nails, it must set, or polymerize, in a period of time which is adequate to permit a skilled nail technician to apply and shape the composition, but not so long as to have the client service become undesirably extended.

Because of these functional use limitations, reduced odor monomers are most commonly employed in beauty schools, where individuals train to become nail technicians. In this environment, the long cure time associated with the reduced odor monomers is actually a benefit because it allows the neophyte technician considerable latitude while developing the necessary application skills. Further, state cosmetology boards require the use of reduced odor monomers during certification exams.

Testing various examples provided in the previous low-odor art reveals that the resulting systems have functional deficiencies. Most often, the ball to be applied to the nail is watery and cannot be properly shaped on the nail. The nail technician typically overcomes this defect by using less liquid on the brush (which makes for a drier mix of a sandy consistency), working in smaller areas and/or allowing for a longer open time/shorter working time of the slurry.

The known low-odor monomer systems also typically provide an unsatisfactory surface. It is necessary for the applied nail coating from the preferred monomer-polymer homogeneous mixture to be fixed and hard within 60 to 600 seconds from the time of initial mixing, after which, the coating can be further modified, such as shaped with a nail file or other shaping tool, embellished and/or covered with a gloss coat or polish. The finished surface of the fingernail enhancement must have no tacky or wet layer present. Otherwise, the surface is capable of transferring to the technician's gloves and fouls any other instruments, powder or nails coming in contact with the surface.

The surface of the finished nail enhancement must be hard to the touch in no more than 10 minutes after the application is completed, and preferably in much less than 10 minutes. For the nail technician, hardness can be easily and qualitatively evaluated by scratching with a fingernail. If too soft, the applied enhancements can be damaged while working on other nails. If too soft, such as is the case when an un-polymerized/oxygen inhibition layer is present, buffing will undesirably result in a gummy residue. In the trade, this residue may be referred to as “roll off”. When the surface is not hard, too much of the enhancement will be removed during shaping, the resultant surface will be rough, the technician will spend excess time cleaning the debris, and/or the buffing equipment (such as an electronic nail file bit) will clog. Preferentially, sanding of the cured enhancement results in a fine powder, making for a smooth nail enhancement surface, with minimal excess removal and easy clean up.

If the setting (hardening or polymerization) is too rapid, the nail technician has insufficient time to sculpt the enhancement. In contrast, if the setting is too slow, the service time can become undesirably long. Known reduced odor systems may eventually cure—in hours—but this time frame is unacceptably long to be practical for nail salon use. A nail salon is motivated to provide attention to each client, while also servicing as many clients as is practical during operating hours (turnover). Overly extended service times both exasperate customers and diminish salon revenue potential.

There are a variety of reasons why the surface of an acrylic nail enhancement may be fluid after 5 minutes and/or not sufficiently hardened after 10 minutes. The literature is replete with explanations of un-polymerized monomers and/or the presence of an oxygen inhibition layer. A known defect associated with (meth) acrylic coatings is that the un-polymerized/inhibition layer must be removed, either with filing or an alcohol wipe. This is an undesirable extra step and what remains is an irregular and thinned film.

U.S. Pat. No. 5,523,076 describes an improvement in the flexural strength of a conventional acrylic nail enhancement by using combinations of ethyl (meth)acrylate (EMA) and 2-hydroxyethyl (meth)acrylate (HEMA) in the monomer liquid. These combinations are used alongside polymers synthesized from EMA, HEMA, MMA and combinations thereof.

Further in U.S. Pat. No. 5,523,076, a system with 70% EMA and 30% HEMA is described. Given the high vapor pressure and unpleasant odor presented by EMA, it would be desirable for any monomer liquid composition containing EMA to contain significantly less than 70% EMA. In the time since the publication of U.S. Pat. No. 5,523,076, the safety of HEMA has come into question. Overuse is thought to promote an excessive allergic reaction. As a result, responsible industry practitioners are avoiding and/or reducing formulation content of the HEMA monomer. Thus, there is still a need to create a monomer liquid combination such that the monomer-polymer binary system is not harmful to the user's natural nail or to any of the tissue surrounding or underlying the nail.

U.S. Pat. No. 5,603,924 teaches the use of various monomer liquid combinations, with the objective of reducing the yellow nail color prevalent in conventional applications. In the examples provided, the primary constituent in the monomer liquids is EMA. Secondary ingredients include ethylene glycol di(meth)acrylate, HEMA, and structurally similar components. The polymer powder is described as a copolymer of methyl (meth)acrylate and ethyl (meth)acrylate.

U.S. Pat. No. 5,738,843 attempts to overcome some of the aforementioned curing deficiencies. The liquid portion of this invention, however, allows for up to 95% of a high solvency monomer such as ethyl (meth)acrylate, thereby foregoing the highly desirable aesthetic of reduced odor. Also, of note in this property, up to 50% of a saturated alcohol compound is included in the monomer recipe, to speed the polymerization time of the applied nail enhancement.

In another method to resolve the strong smell associated with EMA, monomer liquids have been formulated to include a pleasing scent, such as a fragrance, perfume or essential oil. Although a fragrance might distract from or mask the odor to a certain extent, its use could have a negative impact on the formation of the artificial nail, especially with respect to the composition's curing properties and surface properties, compared to the monomer liquids without the added fragrance. The identity of the liquid monomer composition can be varied, with the objective of aligning solubility and curing properties to a given polymer powder. Typically composed of an alkyl (meth)acrylate monomer as the primary constituent, many different combinations of (meth) acrylic monomers can be employed and are well known throughout the salon industry. Such monomer compositions may also include any number of additives, including without limitation, cross linking (meth)acrylate esters, amine accelerator(s), polymerization inhibitors, dyes and/or light stabilizers.

Even in the best performing systems, the monomer liquid portion may not completely solubilize the polymer powder, and as a result, the finished enhancement is hazy in appearance and/or visual defects in the form of bubbles or small spherical globules appear entrained within the artificial nail. In their simplest form, the bubbles are entrained air. It is hypothesized that when the monomer liquid and polymer powder are mixed, small pockets of air are created, and due to the viscosity of the mixed powder and liquid, which starts at 25,000 cPs and rapidly increases to a solid state, the trapped air cannot be released. Ideally, when mixing the polymer powder and monomer liquid, every powder particle is surrounded and is wet out & dissolved. It is possible however, that irregularities in powder particles enable powder packing, thus inhibiting penetration and coating by the monomer. Powder that is incompletely solubilized can aggregate with other partially dry powders and as with air, undesirably affix within the cured nail enhancement. Wettability of the polymer powder is a major field of scientific study, where it has been demonstrated that the wetting is influenced by the surface activity of the powder particle, the surface activity of the liquid, the surface area of the powder, the surface charge of the powder, particle size and particle size distribution of the powder, density of the powder, porosity of the powder, atmospheric conditions and other factors.

A common amine polymerization accelerator used in the monomer-polymer nail enhancement industry is N,N-dimethyl-p-toluidine (DMPT). DMPT is a suspected carcinogen and is listed as such by the California Office of Environmental Health Hazard Assessment (OEHHA). It would therefore be highly desirable to create a monomer-polymer nail enhancement system which, in general, is thought to be less potentially hazardous to the user's health, and more specifically, is free of DMPT.

Polymer powder compositions of the low-odor prior art generally comprise the following ingredients: a (meth)acrylate polymer (such as poly(ethyl (meth)acrylate)) or a copolymer of methyl and ethyl (meth)acrylate, and an organic peroxide polymerization initiator (such as benzoyl peroxide). The polymer powder may contain a variety of additives, such as pigments (e.g., titanium dioxide), secondary polymers (e.g., polyvinyl acetate) and flow modifiers (e.g., fumed silica). The properties of the polymer powder can be adjusted by varying the choice of monomer(s) used to synthesize the polymer or copolymer, modifying the resultant character of the polymer (e.g., the molecular weight of polymer, the ratio of chain elements in a copolymer, etc.), changing the particle size distribution of the polymer powder, and including additives with the polymer powder portion.

Notably, the prior art describes polymer powder compositions based on the methyl (meth)acrylate and/or ethyl (meth)acrylate components. Notably, the prior art describes monomer liquid compositions based either on functionalized methacrylic esters, or a majority portion of EMA. These recipes fail to achieve both limited odor and effective solvation of the polymer powder. The prior art incorporates any number of functionalized monomers, but these combinations typically exhibit insufficient solvating power to adequately dissolve the polymer powder, do not promote industry desired working characteristics and do not result in a hard, non-tacky nail enhancement in the time needed to provide a nail salon service.

In view of the above problems, there is an unmet need to create a nail enhancement polymer that provides improved clarity and reduced visual defects, when used with conventional monomer formulations.

In view of the above problems, there is an unmet need to create a monomer-polymer nail enhancement system with improved use characteristics and is of lower odor than conventional formulations.

### SUMMARY OF THE INVENTION

The present invention describes compositions that are unexpectedly advantageous for preparing artificial nails because they are typically low-odor, self-curing, visually clear after application, and demonstrate desirable working properties valued by the salon industry and/or result in an artificial nail surface containing a reduced number and size of entrained defects.

Low vapor pressure monomers have a limited solvating effect on higher T_{g} polymers, including those polymer powders synthesized from short chain monomers. Preferred working properties of polymer-monomer nail enhancements are accomplished by correct matching of the monomer and polymer components. In a preferred embodiment, the monomer sufficiently dissolves the polymer to achieve each of a desired consistency of the dough-like ball, suitable application characteristics on the nail and utility of the finished nail enhancement.

An aspect of the present invention is a nail-enhancing composition comprising:
a polymer component formed from:
   a first (meth)acrylate ester with an alkyl side chain of 3 or less carbon atoms;
   a second (meth)acrylate ester with a primary, secondary or tertiary alkyl side chain of at least 5 carbon atoms; and
an initiator.

Another aspect of the present invention is a nail-enhancing composition comprising:
a monomer component comprising:
   a first (meth)acrylate ester, where the (meth)acrylate ester is di-, tri-, or multi-functional;
   a second (meth)acrylate ester with an alkyl side chain of 3 or less carbon atoms; and
an accelerator.

Another aspect of the present invention is a nail-enhancing composition comprising:
(i) a polymer component formed from:
   a first (meth)acrylate ester with an alkyl side chain of 3 or less carbon atoms; and
   a second (meth)acrylate ester with a primary, secondary or tertiary alkyl side chain of at least 5 carbon atoms; and
   an initiator;
and
(ii) a monomer component comprising:
   a first (meth)acrylate ester, where the (meth)acrylate ester is di-, tri-, or multi-functional;
   a second (meth)acrylate ester with an alkyl side chain of 3 or less carbon atoms; and
   an accelerator.

In an exemplary embodiment, the nail-enhancing composition is a low-odor composition, wherein for the monomer component, the first (meth)acrylate ester is present in a greater amount by weight compared to the second (meth)acrylate ester.

In an exemplary embodiment of the nail-enhancing composition, for the monomer component, the second (meth)acrylate ester is present in a greater amount by weight compared to the first (meth)acrylate ester.

In an exemplary embodiment of the nail-enhancing composition, the polymer component is a powder.

In an exemplary embodiment of the nail-enhancing composition, the monomer component is a liquid.

In an exemplary embodiment of the nail-enhancing composition, the first (meth)acrylate ester of the monomer component is a di-, tri- or multi-functional (meth)acrylate ester.

In an exemplary embodiment of the nail-enhancing composition, the second (meth)acrylate ester of the monomer component has an alkyl side chain of 3 or less carbon atoms.

In an exemplary embodiment of the nail-enhancing composition, the accelerator is an amine accelerator.

In an exemplary embodiment of the nail-enhancing composition, the composition further comprises a UV absorber.

In an exemplary embodiment of the nail-enhancing composition, the composition further comprises a polymerization inhibitor.

In an exemplary embodiment of the nail-enhancing composition, the composition further comprises a fragrance.

In an exemplary embodiment of the nail-enhancing composition, the composition further comprises one or more dyes, pigments or other colorants.

In an exemplary embodiment of the nail-enhancing composition, the first (meth)acrylate ester of the monomer component is selected from the group consisting of 1,3-butanediol di(meth)acrylate; 1,4-butanediol di(meth)acrylate; 1,6-hexanediol di(meth)acrylate; glycerol di(meth)acrylate; ethylene glycol di(meth)acrylate; diethylene glycol di(meth)acrylate; triethylene glycol di(meth)acrylate; tetraethylene glycol di(meth)acrylate; and combinations thereof.

In an exemplary embodiment of the nail-enhancing composition, the first (meth)acrylate ester of the monomer component is 1,4-butanediol di(meth)acrylate.

In an exemplary embodiment of the nail-enhancing composition, the first (meth)acrylate ester of the monomer component is 1,4-butanediol di(meth)acrylate and the second (meth)acrylate ester is ethyl (meth)acrylate.

In an exemplary embodiment of the nail-enhancing composition, the first (meth)acrylate ester of the monomer component is a blend selected from the group consisting of 1,3-butanediol di(meth)acrylate; 1,4-butanediol di(meth)acrylate; 1,6-hexanediol di(meth)acrylate; glycerol di(meth)acrylate; ethylene glycol di(meth)acrylate; diethylene glycol di(meth)acrylate; triethylene glycol di(meth)acrylate; tetraethylene glycol di(meth)acrylate; and in which blend, 1,4-butanediol di(meth)acrylate is the majority component.

In an exemplary embodiment of the nail-enhancing composition, the amine accelerator is N-(2-hydroxyethyl)-N-methyl-para-toluidine.

In an exemplary embodiment of the nail-enhancing composition, the amine accelerator is poly(oxy-1,2-ethanediyl), α,α′-(((4-methylphenyl)imino)di-2,1-ethanediyl)bis($2-hydroxy- or N-methyl-N-(2-hydroxypropyl)-p-toluidine.

In an exemplary embodiment of the nail-enhancing composition, the monomer component has a theoretical vapor pressure of less than 10.0 mmHg at 20° C.

In an exemplary embodiment of the nail-enhancing composition, the ASTM E-679-04 dynamic dilution olfactometry odor concentration of the monomer liquid component is no more than 30,000 OU m³.

In an exemplary embodiment of the nail-enhancing composition, the polymer component is formed from 1 percent to 40 percent of a second (meth)acrylate ester with a primary, secondary or tertiary alkyl side chain of at least 5 carbon atoms.

In an exemplary embodiment of the nail-enhancing composition, the initiator is a peroxide.

In an exemplary embodiment of the nail-enhancing composition, the first (meth)acrylate ester of the polymer component is ethyl (meth)acrylate.

In an exemplary embodiment of the nail-enhancing composition, the second (meth)acrylate ester of the polymer component is 2-ethylhexyl (meth)acrylate.

In an exemplary embodiment of the nail-enhancing composition, the initiator is benzoyl peroxide.

In an exemplary embodiment of the nail-enhancing composition, the use ratio of the monomer component to the polymer component is from 1:3 to 2:1 by weight.

In an exemplary embodiment of the nail-enhancing composition, the polymer component and the monomer component form a single homogeneous phase after mixing.

In an exemplary embodiment of the nail-enhancing composition, the resulting nail enhancement has a glass transition temperature greater than 50° C.

In an exemplary embodiment of the nail-enhancing composition, the polymer component comprises poly(ethyl-co-2-ethylhexyl) (meth)acrylate and benzoyl peroxide; and the monomer component comprises 1,4-butanediol di(meth)acrylate, ethyl (meth)acrylate and N-methyl-N-(2-hydroxyethyl)-p-toluidine.

In an exemplary embodiment of the nail-enhancing composition, the polymer component comprises poly(ethyl-co-2-ethylhexyl) (meth)acrylate and benzoyl peroxide; and the majority monomer component is ethyl (meth)acrylate.

In an exemplary embodiment of the nail-enhancing composition, the composition further comprises one or more of a fragrance, a colorant, a plasticizer, a polymerization inhibitor and/or a UV absorber.

In an exemplary embodiment of the nail-enhancing composition, the first (meth)acrylate ester of the polymer component is ethyl (meth)acrylate and is present in an amount of no less than 60 weight percent relative to the total weight of the polymer component.

Another aspect of the present invention is a method of forming an artificial nail enhancement, comprising:
applying the composition of any one of exemplary embodiments to a human nail;
forming from the applied composition a coating and/or artificial extension on a surface of the nail;
optionally extending the artificial nail beyond the length of the nail; and
optionally buffing of the artificial nail to smooth its surface.

Another aspect of the present invention is a method of forming an artificial nail enhancement, comprising:
wetting an applicator in a monomer liquid, the liquid comprising:
   a first (meth)acrylate ester, where the (meth)acrylate ester is di-, tri-, or multi-functional;
   a second (meth)acrylate ester with an alkyl side chain of 3 or less carbon atoms; and
   an accelerator;
dipping the wetted applicator into a polymer powder for a time sufficient to allow the monomer on the applicator to mix with and absorb the polymer powder, where the polymer powder is formed from:
   a first (meth)acrylate ester with an alkyl side chain of 3 or less carbon atoms;
   a second (meth)acrylate ester with a primary, secondary or tertiary alkyl side chain of at least 5 carbon atoms; and
   an initiator;
removing the applicator from the powder and allowing the resulting heterogeneous mixture present on the applicator to transition from two phases into one homogeneous mixture;
transferring the homogeneous mixture to a surface of a human nail;
shaping the mixture with the applicator to form a coating and/or artificial nail extension;
optionally extending an artificial nail beyond the length of the nail; and
optionally buffing the artificial nail to smooth its surface.

In an exemplary embodiment of the method of forming an artificial nail enhancement, the use ratio of the monomer liquid to the polymer powder is from 1:3 to 2:1 by weight.

In an exemplary embodiment of the method of forming an artificial nail enhancement, the monomer liquid and polymer powder cure into a hardened film in no less than 60 seconds and no more than 600 seconds after mixing.

In an exemplary embodiment of the method of forming an artificial nail enhancement, the finished nail enhancement has an ASTM D-1003 transmission haze of no more than 40 candela.

In an exemplary embodiment of the method of forming an artificial nail enhancement, 10 minutes after application, the finished nail enhancement has an ASTM D-2240 Shore “D” hardness of greater than 60.0.

An aspect of the present invention is an artificial nail kit containing
(i) a polymer component, where the polymer component is formed from:
   a first (meth)acrylate ester with an alkyl side chain of 3 or less carbon atoms;
   a second (meth)acrylate ester with a primary, secondary or tertiary alkyl side chain of at least 5 carbon atoms; and
   a polymerization initiator;
(ii) a monomer component comprising:
   a first (meth)acrylate ester, where the (meth)acrylate ester is di-, tri- or multi-functional;
   a second (meth)acrylate ester with an alkyl side chain of 3 or less carbon atoms; and
   an accelerator; and
(iii) instructions for use.

An aspect of the present invention is a human nail modified by the artificial nail kit as described herein.

In an exemplary embodiment of the nail-enhancing composition, the second (meth)acrylate ester of the monomer component is ethyl (meth)acrylate which is present in an amount of less than 50 weight percent relative to the total weight of the monomer component.

In an exemplary embodiment of the nail-enhancing composition, the second (meth)acrylate ester of the monomer component is ethyl (meth)acrylate which is present in an amount of greater than 50 weight percent relative to the total weight of the monomer component.

In an exemplary embodiment of the nail-enhancing composition, the first (meth)acrylate ester of the polymer component is ethyl (meth)acrylate which is present in an amount of no less than 60 weight percent relative to the total weight of the polymer component, and the second (meth)acrylate ester of the monomer component is ethyl (meth)acrylate which is present in an amount of less than 50 weight percent relative to the total weight of the monomer component.

In an exemplary embodiment of the nail-enhancing composition, the first (meth)acrylate ester of the polymer component is ethyl (meth)acrylate which is present in an amount of no less than 60 weight percent relative to the total weight of the polymer component, and the second (meth)acrylate ester of the monomer component is ethyl (meth)acrylate which is present in an amount of greater than 50 weight percent relative to the total weight of the monomer component.

A preferred exemplary embodiment of the nail-enhancing composition (referred to hereinafter as “preferred composition 1”), is a nail-enhancing, low-odor composition comprising:
(i) a polymer component formed from:
   a first (meth)acrylate ester with an alkyl side chain of 3 or less carbon atoms; and
   a second (meth)acrylate ester with a primary, secondary or tertiary alkyl side chain of at least 5 carbon atoms; and
   an initiator;
(ii) a monomer component comprising:
   a first (meth)acrylate ester, where the (meth)acrylate ester is di-, tri- or multi-functional; and
   a second (meth)acrylate ester with an alkyl side chain of 3 or less carbon atoms; and
   an accelerator,
   wherein for the monomer component, the first (meth)acrylate ester is present in a greater amount by weight compared to the second (meth)acrylate ester.

Another preferred exemplary embodiment of the nail-enhancing composition (referred to hereinafter as “preferred composition 2”), is a nail-enhancing composition comprising:
(i) a polymer component formed from:
   a first (meth)acrylate ester with an alkyl side chain of 3 or less carbon atoms; and
   a second (meth)acrylate ester with a primary, secondary or tertiary alkyl side chain of at least 5 carbon atoms; and
   an initiator,
(ii) a monomer component comprising:
   a first (meth)acrylate ester, where the (meth)acrylate ester is di-, tri- or multi-functional; and
   a second (meth)acrylate ester with an alkyl side chain of 3 or less carbon atoms; and
   an accelerator,
   wherein for the monomer component, the second (meth)acrylate ester is present in a greater amount by weight compared to the first (meth)acrylate ester.

In an exemplary embodiment of the nail-enhancing composition, such as in, for example, preferred composition 1 and preferred composition 2, the alkyl side chain of the first (meth)acrylate ester of the polymer component contains 3 carbons.

In an exemplary embodiment of the nail-enhancing composition, such as in, for example, preferred composition 1 and preferred composition 2, the alkyl side chain of the first (meth)acrylate ester of the polymer component contains 2 carbons.

In an exemplary embodiment of the nail-enhancing composition, such as in, for example, preferred composition 1 and preferred composition 2, the alkyl side chain of the first (meth)acrylate ester of the polymer component contains 1 carbon.

In an exemplary embodiment of the nail-enhancing composition, such as in, for example, preferred composition 1 and preferred composition 2, the alkyl side chain of the second (meth)acrylate ester of the polymer component is a primary side chain.

In an exemplary embodiment of the nail-enhancing composition, such as in, for example, preferred composition 1 and preferred composition 2, the alkyl side chain of the second (meth)acrylate ester of the polymer component is a secondary side chain.

In an exemplary embodiment of the nail-enhancing composition, such as in, for example, preferred composition 1 and preferred composition 2, the alkyl side chain of the second (meth)acrylate ester of the polymer component is a tertiary side chain.

In an exemplary embodiment of the nail-enhancing composition, such as in, for example, preferred composition 1 and preferred composition 2, the alkyl side chain of the second (meth)acrylate ester of the polymer component is at least 5 carbon atoms, but no more than 18 carbon atoms, such as at least 5 carbon atoms, but no more than 15 carbon atoms, such as at least 5 carbon atoms but no more than 10 carbon atoms.

In an exemplary embodiment of the nail-enhancing composition, such as in, for example, preferred composition 1 and preferred composition 2, the polymer component is in solid form, such as a powder.

In an exemplary embodiment of the nail-enhancing composition, such as in, for example, preferred composition 1 and preferred composition 2, the monomer component is a liquid.

In an exemplary embodiment of the nail-enhancing composition, such as in, for example, preferred composition 1 and preferred composition 2, the first (meth)acrylate ester of the monomer component is a di-, tri- or multi-functional (meth)acrylate ester.

In an exemplary embodiment of the nail-enhancing composition, such as in, for example, preferred composition 1 and preferred composition 2, the first (meth)acrylate ester of the monomer component is di-functional.

In an exemplary embodiment of the nail-enhancing composition, such as in, for example, preferred composition 1 and preferred composition 2, the first (meth)acrylate ester of the monomer component is tri-functional.

In an exemplary embodiment of the nail-enhancing composition, such as in, for example, preferred composition 1 and preferred composition 2, the first (meth)acrylate ester of the monomer component is multi-functional.

In an exemplary embodiment of the nail-enhancing composition, such as in, for example, preferred composition 1 and preferred composition 2, the second (meth)acrylate ester of the monomer component has an alkyl side chain of less than 3 carbon atoms.

In an exemplary embodiment of the nail-enhancing composition, such as in, for example, preferred composition 1 and preferred composition 2, the alkyl side chain of the second (meth)acrylate ester of the monomer component contains 3 carbons.

In an exemplary embodiment of the nail-enhancing composition, such as in, for example, preferred composition 1 and preferred composition 2, the alkyl side chain of the second (meth)acrylate ester of the monomer component contains 2 carbons.

In an exemplary embodiment of the nail-enhancing composition, such as in, for example, preferred composition 1 and preferred composition 2, the alkyl side chain of the second (meth)acrylate ester of the monomer component contains 1 carbon.

In an exemplary embodiment of the nail-enhancing composition, such as in, for example, preferred composition 1 and preferred composition 2, the accelerator is an amine accelerator.

In an exemplary embodiment of the nail-enhancing composition, such as in, for example, preferred composition 1 and preferred composition 2, the accelerator is a barbituric acid.

In an exemplary embodiment of the nail-enhancing composition, such as in, for example, preferred composition 1 and preferred composition 2, the composition further comprises a UV absorber.

In an exemplary embodiment of the nail-enhancing composition, such as in, for example, preferred composition 1 and preferred composition 2, the composition further comprises a polymerization inhibitor.

In an exemplary embodiment of the nail-enhancing composition, such as in, for example, preferred composition 1 and preferred composition 2, the composition further comprises one or more dyes, pigments or other colorants.

In an exemplary embodiment of the nail-enhancing composition, such as in, for example, preferred composition 1 and preferred composition 2, the first (meth)acrylate ester of the monomer component is selected from the group consisting of 1,3-butanediol di(meth)acrylate; 1,4-butanediol di(meth)acrylate; 1,6-hexanediol di(meth)acrylate; glycerol di(meth)acrylate; ethylene glycol di(meth)acrylate; diethylene glycol di(meth)acrylate; triethylene glycol di(meth)acrylate; tetraethylene glycol di(meth)acrylate; and combinations thereof.

In an exemplary embodiment of the nail-enhancing composition, such as in, for example, preferred composition 1, the first (meth)acrylate ester of the monomer component is 1,4-butanediol di(meth)acrylate.

In an exemplary embodiment of the nail-enhancing composition, such as in preferred composition 1, the first (meth)acrylate ester of the monomer component is 1,4-butanediol di(meth)acrylate, the second (meth)acrylate ester is ethyl (meth)acrylate and a third component is a plasticizer.

In an exemplary embodiment of the nail-enhancing composition, such as in, for example, preferred composition 1 and preferred composition 2, the amine accelerator is N-(2-hydroxyethyl)-N-methyl-para-toluidine.

In an exemplary embodiment of the nail-enhancing composition, such as in, for example, preferred composition 1 and preferred composition 2, the amine accelerator is poly(oxy-1,2-ethanediyl), α,α′-(((4-methylphenyl)imino)di-2,1-ethanediyl)bis(Ω-hydroxy- or N-methyl-N-(2-hydroxypropyl)-p-toluidine.

In an exemplary embodiment of the nail-enhancing composition, such as in, for example, preferred composition 1, the vapor pressure of the monomer liquid component is less than 10.0 mmHg at 20° C.

In an exemplary embodiment of the nail-enhancing composition, such as in, for example, preferred composition 1, the ASTM E-679-04 dynamic dilution olfactometry odor concentration of the monomer liquid component is no more than 30,000 OU m³.

In an exemplary embodiment of the nail-enhancing composition, the polymer component is formed from 1 percent to 40 percent of a second (meth)acrylate ester with a primary, secondary or tertiary alkyl side chain of at least 5 carbon atoms.

In an exemplary embodiment of the nail-enhancing composition, the Initiator is an organic or inorganic peroxide.

In an exemplary embodiment of the nail-enhancing composition, the polymer component comprises poly(ethyl-co-2-ethylhexyl) (meth)acrylate.

In an exemplary embodiment of the nail-enhancing composition, poly(ethyl-co-2-ethylhexyl) (meth)acrylate is the only (meth)acrylate-based polymer present in the polymer component.

In an exemplary embodiment of the nail-enhancing composition, the initiator is an organic peroxide.

In an exemplary embodiment of the nail-enhancing composition, the initiator is benzoyl peroxide.

In an exemplary embodiment of the use method, the ratio between the monomer component to the polymer component is from 1:3 to 2:1 by weight.

In an exemplary embodiment of the nail-enhancing composition, the polymer component and the monomer component form a homogeneous mixture.

In an exemplary embodiment of the nail-enhancing composition, the polymer component of the composition is formed from 1-99% by weight of the first (meth)acrylate ester with an alkyl side chain of 3 or less carbon atoms and 1-50% by weight of the second (meth)acrylate ester with a primary, secondary or tertiary alkyl side chain of at least 5 carbon atoms and between 0 to 5% by weight of the initiator.

In an exemplary embodiment of the nail-enhancing composition, the monomer component of the composition comprises 10-90% by weight of the first (meth)acrylate ester, where the (meth)acrylate ester is di-, tri-, or multi-functional and 10-90% by weight of the second (meth)acrylate ester with an alkyl side chain of 3 or less carbon atoms and between 0 to 5% by weight of the accelerator.

In an exemplary embodiment, the nail-enhancing, low-odor composition comprises: a monomer component comprising 1,4 butanediol di(meth)acrylate, ethyl (meth)acrylate and N-methyl-N-(2-hydroxyethyl)-p-toluidine; a polymer component comprising poly(ethyl-co-2-ethylhexyl (meth)acrylate); and benzoyl peroxide.

In an exemplary embodiment, the nail-enhancing, low-odor composition comprises (or alternatively, consists of):
a monomer component comprising (or alternatively, consisting of) 1,4 butanediol di(meth)acrylate, ethyl (meth)acrylate and N-methyl-N-(2-hydroxyethyl)-p-toluidine; and one or more of a fragrance, a colorant (such as a dye), and a UV light absorber; and
a polymer component comprising (or alternatively, consisting of) poly(ethyl-co-2-ethylhexyl) (meth)acrylate, benzoyl peroxide and one or more colorants.

In the preferred composition 2, the polymer component is a poly(meth)acrylate copolymer, which preferentially dissolves in commercial monomers. The dissolution is such that the polymer component, which is typically in powder form, readily absorbs the monomer liquid, resulting in a nail enhancement substantially free of defects.

In the preferred composition 2, the polymer component is a poly(meth)acrylate copolymer, which preferentially is used without any changes to the application techniques that are preferred by nail technicians.

In the preferred composition 2, the poly(meth)acrylate is a copolymer.

In the preferred composition 2, the poly(meth)acrylate is a solid in powder form.

In an exemplary embodiment of the preferred composition 1, the composition becomes homogeneous upon mixing of the solid, poly(meth)acrylate component and a low-odor liquid monomer component.

In an exemplary embodiment of the preferred composition 2, the composition becomes homogeneous upon mixing of the solid, poly(meth)acrylate component and a commercial liquid monomer component.

In an exemplary embodiment of the preferred composition 1 and the preferred composition 2, the composition includes a polymer component made from:
a first (meth)acrylate ester with an alkyl side chain of 3 or less carbon atoms;
a second (meth)acrylate ester with a primary, secondary or tertiary alkyl side chain of at least 5 carbon atoms; and
an initiator.

In an exemplary embodiment of the preferred composition 1 and the preferred composition 2, a preferred first (meth)acrylate ester of the polymer component is ethyl (meth)acrylate.

In an exemplary embodiment of the preferred composition 1 and the preferred composition 2, a preferred first (meth)acrylate ester of the polymer component is methyl (meth)acrylate.

In an exemplary embodiment of the preferred composition 1 and the preferred composition 2, the alkyl side chain of the first (meth)acrylate ester of the polymer component preferably contains 3 or less carbons.

In an exemplary embodiment of the preferred composition 1 and the preferred composition 2, the alkyl side chain of the second (meth)acrylate ester of the polymer component preferably is a primary side chain.

In an exemplary embodiment of the preferred composition 1 and the preferred composition 2, the alkyl side chain of the second (meth)acrylate ester of the polymer component is preferably a secondary side chain.

In an exemplary embodiment of the preferred composition 1 and the preferred composition 2, the alkyl side chain of the second (meth)acrylate ester of the polymer component is preferably a tertiary side chain.

In an exemplary embodiment of the preferred composition 1 and the preferred composition 2, the alkyl side chain of the second (meth)acrylate ester of the polymer component is preferably at least 5 carbon atoms, but no more than 18 carbon atoms, such as at least 5 carbon atoms, but no more than 15 carbon atoms, such as at least 5 carbon atoms but no more than 10 carbon atoms.

In an exemplary embodiment of the preferred composition 1 and the preferred composition 2, the polymer component is preferably formed from 1 percent to 40 percent of a second (meth)acrylate ester with a primary, secondary or tertiary alkyl side chain of at least 5 carbon atoms.

In an exemplary embodiment of the preferred composition 1 and the preferred composition 2, the initiator is preferably an organic or inorganic peroxide.

In an exemplary embodiment of the preferred composition 1 and the preferred composition 2, the polymer component preferably comprises poly(ethyl-co-2-ethylhexyl) (meth)acrylate.

In an exemplary embodiment of the preferred composition 1 and the preferred composition 2, poly(ethyl-co-2-ethylhexyl) (meth)acrylate is preferably the only (meth)acrylate-based polymer present in the polymer component.

In an exemplary embodiment of the preferred composition 1 and the preferred composition 2, the initiator is preferably an organic peroxide.

In an exemplary embodiment of the preferred composition 1 and the preferred composition 2, the initiator is preferably benzoyl peroxide.

In an exemplary embodiment of the preferred composition 1 and the preferred composition 2, the polymer component of the composition is preferably formed from 1-99% by weight of the first (meth)acrylate ester with an alkyl side chain of 3 or less carbon atoms and 1-50% by weight of the second (meth)acrylate ester with a primary, secondary or tertiary alkyl side chain of at least 5 carbon atoms and between 0 to 5% by weight of the initiator.

In the preferred composition 2, the polymer powder of the invention is used in combination with a commercial monomer component, that is comprised of 50% or more ethyl (meth)acrylate.

An exemplary embodiment of using the preferred composition 1 and the preferred composition 2 is to make an artificial nail enhancement by the following:
wetting an applicator in a monomer liquid by dipping the wetted applicator into a polymer powder for a time sufficient to allow the monomer on the applicator to mix with and absorb the polymer powder, where the polymer powder is formed from:
   a first (meth)acrylate ester with an alkyl side chain of 3 or less carbon atoms;
   a second (meth)acrylate ester with a primary, secondary or tertiary alkyl side chain of at least 5 carbon atoms;
   and an initiator;
allowing the resulting heterogeneous mixture present on the applicator to transition from two phases into one homogeneous mixture;
transferring the homogeneous mixture to a surface of a human nail;
shaping the mixture with the applicator to form a coating and/or artificial nail extension;
optionally extending an artificial nail beyond the length of the nail; and
optionally buffing the artificial nail to smooth its surface.

In an exemplary embodiment when applying the preferred composition 1 and preferred composition 2 of the invention, the use ratio of the monomer liquid to the polymer powder is from 1:3 to 2:1 by weight.

In an exemplary embodiment using the polymer powder of the preferred composition 1 and the preferred composition 2, the respective monomer liquid and polymer powder cure into a hardened film in no less than 60 seconds and no more than 600 seconds after mixing.

In an exemplary embodiment of the preferred composition 1 and the preferred composition 2, the nail enhancement resulting from a respective monomer liquid and polymer powder has a glass transition temperature no less than 50° C.

In an exemplary embodiment of the preferred composition 1 and the preferred composition 2, the nail enhancement polymer component is comprised of (or alternatively, consists of) poly(ethyl-co-2-ethylhexyl) (meth)acrylate, benzoyl peroxide and one or more colorants.

### BRIEF DESCRIPTION OF THE DRAWINGS

The figures stand for specific embodiments of the invention and are not intended to otherwise limit the scope of the invention as described herein.

FIG. 1 illustrates a comparison of the clarity of several low-odor and conventional nail applications compared with a nail application of the present application.

FIG. 2 illustrates the results of Tables 12 and 13.

FIG. 3 illustrates the results of Table 14.

FIG. 4 illustrates the results of Tables 15 and 16.

FIG. 5 illustrates the results of Table 17.

### DETAILED DESCRIPTION OF THE INVENTION

The terms “nail” and “nail surface” as used here refer to the natural, keratinaceous nail surface. The compositions of the invention may be applied directly to the keratinaceous surface of a natural nail (e.g., fingernail, toenail) of a mammal such as a human being.

The term “ball” as used herein refers to an agglomerated form of the mixture of the polymer component and the monomer component of the composition of the invention, typically prior to application of the composition to the nail surface and is not limited to a spherical shape.

The phrase “nail coating” and “nail enhancement” as used here refers to a hardened, cured material covering all or a portion of the nail surface, and to any portions of the material that extends beyond the edge of the nail.

The term “poly((meth)acrylate) component” as used herein refers to a copolymer, a terpolymer or a tetramer.

In an embodiment, the invention relates to reduced odor compositions that are suitable for making artificial nails and/or decorative coatings on human fingernails and toenails, where the compositions represent a combination of a monomer liquid and polymer powder (a nail enhancement system), which, upon being mixed at the time of use, polymerize to a hard, fused polymer in the shape of an artificial nail and/or working characteristics are unexpectedly achieved only from the combination of the monomer liquid and polymer powder.

In an embodiment the invention relates to compositions that may be, but are not required to be of reduced odor, where the nature of the claimed nail enhancement system is such that desirable characteristics of reduced visual defects are unexpectedly achieved only from the polymer powder.

The essential failing of the prior art is that the employed polymer powders are typically prepared from short alkyl chain monomers, where the alkyl chain ranges from 1 or 2 carbon atoms (i.e., methyl (meth)acrylate and/or ethyl (meth)acrylate). It is hypothesized that the functional (meth)acrylate monomers used within industry may not completely wet or dissolve the polymer powder, leaving visual defects in the resultant nail enhancement. As a result, it is possible that modification of the polymer powder, to more readily dissolve in established monomer liquids, could serve to resolve some or all of the visual defects observed in the resultant nail enhancement.

In an exemplary embodiment of the preferred composition 2, the composition consists of a polymer powder portion and a monomer liquid portion, of which, many commercial monomer liquids are suitable.

In exemplary embodiment of the preferred composition 2, the polymer powder is used in combination with a commercial monomer liquid to prepare an acrylic nail enhancement, during which process, the technique and characteristics of application are unchanged for the nail technician.

These two (polymer powder and liquid monomer) portions are combined at the time of use to apply, shape and form an artificial nail/coating. A preferred ratio of the monomer liquid portion to the polymer powder portion is from about 1:3 to about 2:1 by weight, and more preferably from about 1:3 to about 1:1 by weight, such as about 1:2 to about 2:1 by weight, such as about 1:1 to 2:1 by weight. In an exemplary embodiment, the monomer liquid portion-to-polymer powder portion ratio can be managed by relationship need to generate a more or less readily malleable or fluid ball for application to the nail.

In mixing the monomer liquid with the polymer powder, a free-radical polymerization process is started. Without being bound by any theory or hypothesis, the presently accepted mechanism for free radical polymerization for (meth)acrylates is as follows: the first step forms the free radical species which reacts with a (meth) acrylic monomer in a process called initiation. Typically, the free radical species attacks the less sterically hindered carbonyl atom of the (meth)acrylate molecule to form a more stable carbon-centered radical which in turn attacks the double bond of a nearby (meth)acrylate monomer to form an energized dimer which in turn reacts with another monomer to form an energized trimer and so on. This well-known progressive addition of new monomers to the growing oligomer/polymer chain is termed chain propagation, creating a head-to-tail arrangement of monomers into chains.

Thus, as the initiator, (e.g., an organic peroxide), which is present in an exemplary embodiment in the polymer powder, dissolves in the monomer once the polymer powder and the monomer liquid are mixed, it produces free radicals through interaction with the accelerator (e.g., an amine accelerator) that is already present in the monomer. Organic peroxides hold a weak covalent peroxide bond located between the two oxygen atoms that, when exposed to suitable conditions, readily and symmetrically cleaves to yield two individual highly energetic species which are relatively stable carbon-centered radicals. For example, upon decomposition, benzoyl peroxide yields benzoyloxyl radicals, which initiate polymerization.

Non-limiting examples of suitable polymerization initiators include azo, peroxy and redox initiators. In an exemplary embodiment, the initiators may be selected from one or more of benzoyl peroxide, dicumyl peroxide, tert-butyl peroxide, cumene hydroperoxide, diacetyl peroxide, acetyl acetone peroxide, ascaridole, di-(1-napthoyl) peroxide, methyl ethyl ketone peroxide and barbituric acid (2,4,6 (1H,3H,5H)pyrimidine trione) (see U.S. Pat. No. 6,080,389). Other known radical initiators may also be suitable for use. A preferred peroxide initiator is benzoyl peroxide, which in a particular embodiment is present in a finely divided form, and well dispersed or blended in the polymer powder portion. In a preferred embodiment, the initiator is present at from about 0.1 percent to about 3.0 percent by weight of the polymer powder.

As an alternative to the chemical cure mechanism that is known in the nail industry, polymerization of the inventive composition may also be accomplished by exposure of the uncured compositions to radiant energy, such as heat or ultraviolet (UV) light. In various embodiments, photoinitiators, such as, UV electromagnetic radiation (with wavelengths ranging from, for example, 10 to 400 nanometers) photoinitiators, visible electromagnetic radiation (with wavelengths ranging from, for example, 400 to 700 nanometers) photoinitiators, UV-A (ultraviolet A) with wavelengths ranging from, for example, 315 to 400 nanometers) photoinitiators and polychromatic (i.e., both UV and visible light) photoinitiators are incorporated into the monomer liquid.

The photoinitiators described herein may be used alone or in combination. In an exemplary embodiment, a catalyst or synergist may be used to accelerate photopolymerization.

Many of the photoinitiators are well known in the art and may include, without limitation, any type of Norrish Type I or Type II photoinitiator which, upon UV/Vis irradiation or polychromatic irradiation, forms free radicals, including but not limited to benzoins and substituted benzoins, including benzoin, benzoin ether derivatives, such as benzoin methyl ether, benzoin ethyl ether and benzoin isopropyl ether, benzoin phenyl ether and benzoin acetate, acetophenones, including acetophenone, 2,2-dimethoxyacetophenone and 1,1-dichloroacetophenone, benzyl, benzyl formats, benzyl ketals, such as benzyl dimethyl ketal and benzyl diethyl ketal, anthraquinones, including 2-methylanthraquinone, 2-ethylanthraquinone, 2-tert-butylanthraquinone, 1-chloroanthraquinone and 2-amylanthraquinone, triphenylphosphine, acylphosphine oxides, amino acetophenones, benzoylphosphine oxides for example 2,4,6-trimethylbenzoyldiphenylphosphine oxide, benzophenones, such as benzophenone and 4,4′-bis(N,N′-dimethylamino)benzophenone, thioxanthones such as isopropyl thioxanthones and xanthones, acridine derivatives, phenazine derivatives, quinoxaline derivatives or 1-phenyl-1,2-propanedione, 2-O-benzoyl oxime, alpha amino ketones and hydroxyalkyl ketones such as 1-aminophenyl ketones or 1-hydroxyphenyl ketones, such as 1-hydroxycyclohexyl phenyl ketone, phenyl 1-hydroxyisopropyl ketone and 4-isopropylphenyl 1-hydroxyisopropyl ketone, all of which are known compounds.

Some non-limiting examples of UV/Vis photopolymerizable initiators are 2-isopropyl thioxanthone, or 2,2-dimethoxy-1,2-diphenylethan-1-one, or 2-benzyl-2-(dimethylamino)-1-[4-(morpholinyl)phenyl]-1-butanone, or bis(2,4,6,trimethylbenzoyl)-phenyphosphinate, or 2-methyl-1[4-(methylthio)phenyl]-2-morpholinopropan-1-one, benzophenone, 2-dimethylamino-2-(4-methyl-benzyl)-1-(4-morpholin-4-yl-phenyl)-butan-1-one, or -1-hydroxycyclohexyl phenyl ketone, or -2-hydroxy-2-methyl-phenyl-propane-1-one, or 1-[4-(2-hydroxyethoxy)-phenyl]-2-hydroxy-2-methyl-1-propane-1-one, or 2,4,6-trimethylbenzoyldiphenylphosphine oxide, or ethyl (2,4,6-trimethylbenzoyl) phenyl phosphinate, or 2,2-dimethoxy-2-phenylacetophenone, and camphorquinone and benzophenone all of which are known compounds.

When activating polymerization with a photoinitiator, the slurry composition of monomer liquid and polymer powder is applied to nails and shaped in the desired configuration. The coated nails are then exposed to radiant energy, and polymerization occurs.

A combination of the initiator and the accelerator, in addition to the primary constituents of the monomer and polymer components of the composition, allows sufficient time to shape the resulting mixture of the monomer and polymer components into the desired form upon application to the nail, but also results in polymerization (solidification) of the mixture within a reasonable length of time, i.e., during and after application of the mixture to the nail.

Acceleration of reaction times can be achieved by using excess initiator and/or accelerator, but this is typically undesirable because it can result in a rapid and unacceptable heat spike (exotherm) during the curing process and may actually burn the nail and surrounding tissue. When excess initiator and/or accelerator is present, the resultant nail enhancement is weak and impractical for wear. Excess initiator and/or accelerator will also discolor from overage of the amine accelerator, compounding the already yellow color tendencies of previous reduced odor monomers. It is thus desirable for a monomer liquid-polymer powder mixture to begin polymerizing rapidly, such as, for example, in no more than 10 seconds (such as no more than 5 seconds) from the time of the initial monomer-polymer mixing. It is desirable for the monomer-polymer mixture to exhibit only a mild exotherm, at a minimum to avoid user discomfort and also to avoid the generation of excessive heat during cure.

### Polymer Component

The polymer component, typically in the form of a solid, such as a powder, is synthesized from one or more (meth)acrylate monomers. In the nail enhancement industry, known monomer inputs include ethyl (meth)acrylate, methyl (meth)acrylate, and combinations thereof. These finely divided polymers or copolymers in particulate form (directly synthesized by techniques known in industry or by similar means for generating the desired particulate form) generally comprise 80% to 99.9% by weight (such as 85 to 99.9%, such as 90 to 99.9%, such as 85 to 95%, such as 90 to 95%) of the polymer powder portion of the acrylic nail enhancement system. In industry, the polymer particle size Mv is typically close to 60 microns but the polymer powders of the present invention are not so limited. In an exemplary embodiment, the particle size Mv ranges from 1 to 200 microns, such as 1 to 100 microns, such as 30 to 150 microns, such as 40 to 130 microns, such as 50 to 100 microns, such as 60 to 100 microns, such as 70 to 100 microns, such as 80 to 100 microns.

In an exemplary embodiment, the polymer component of the present invention is a poly(meth)acrylate copolymer that is comprised of the following ingredients, based upon the dry weight of polymer component:
a) from 1 to 99%, such as 5 to 95%, such as 10 to 90%, such as 10 to 80%, such as 20 to 95%, such as 20 to 90%, such as 30 to 90%, such as 40 to 90%, such as 50 to 90% of a (meth)acrylate ester having 3 or less carbon atoms in the alkyl side chain, and
b) from 1 to 99%, such as 5 to 95%, such as 10 to 90%, such as 20 to 90%, such as 5 to 80%, such as 10 to 80%, such as 10 to 70%, such as 10 to 60%, such as 10 to 50% of a (meth)acrylate ester having a primary, secondary or tertiary alkyl side chain length of at least 5 carbon atoms, and no more than 18 carbon atoms, and
c) a polymerization initiator, typically present in the polymer component (for initiating the free-radical polymerization of the monomer component/polymer component upon their mixing) in an amount of between 0 and up to 5%, such as 0.05 to 5%, such as 0.1 to 5%, such as 0.05 to 3%, such as 0.1 to 3%.

Usually, monomer liquids need to contain a large concentration of EMA in order to solubilize the EMA polymer, homopolymer or copolymer. Thus, a major deficiency associated with reduced odor monomers is their inability to solubilize industry established polymer powders.

The (meth)acrylate polymer powder compositions of this invention are unique because they are partially synthesized from (meth) acrylic esters with a primary, secondary or tertiary alkyl side chain length of at least 5 carbon atoms. Compared to structures having shorter chain lengths, such as methyl and ethyl (meth)acrylates, it is believed the longer and/or branched structures more effectively enhance the molecular surface area available and are thereby more soluble in a given monomer liquid.

In addition to the below ingredients, the polymer powder portion of the inventive composition may optionally contain pigments (e.g., titanium dioxide), secondary polymers (e.g., finely divided poly(vinyl acetate)), fillers (e.g., hydrated alumina, finely divided glass powder or silicon dioxide), flow modifiers (e.g. fumed silica), colorants, dyes, whiteners, fragrances, stabilizers and antioxidants.

Formulation of the polymer powder is not limited to two co-monomers and may include three, four or more co-monomers. Non-limiting (meth)acrylates which are suitable for preparing the co-polymer powder include alkyl esters of methacrylic acid such as ethyl (meth)acrylate, butyl (meth)acrylate, 2-ethylhexyl (meth)acrylate, etc.; aromatic esters of methacrylic acid such as phenyl (meth)acrylate, benzyl (meth)acrylate, naphthyl (meth)acrylate, etc.; substituted aromatic esters of methacrylic acid such as fluorophenyl (meth)acrylate, chlorophenyl (meth)acrylate, bromophenyl (meth)acrylate, fluorobenzyl (meth)acrylate, chlorobenzyl (meth)acrylate, bromobenzyl (meth)acrylate, etc.; halogenated alkyl esters of methacrylic acid such as fluoromethyl (meth)acrylate, fluoroethyl (meth)acrylate, chloroethyl (meth)acrylate, bromoethyl (meth)acrylate, etc.; methacrylic acid esters such as hydroxyalkyl (meth)acrylates, glycidyl (meth)acrylate, ethylene glycol (meth)acrylate, polyethylene glycol (meth)acrylates, alkylaminoalkyl (meth)acrylate, cyanoalkyl (meth)acrylates, etc.; α-substituted acrylic acid esters such as α-fluoroacrylic acid ester, α-chloroacrylic acid ester, a-cyanoacrylic ester, etc.; methacrylic acid cyclic alkyl esters such as cyclopentyl (meth)acrylate, cyclohexyl (meth)acrylate, methylcyclohexyl (meth)acrylate, trimethylcyclohexyl (meth)acrylate, norbornyl (meth)acrylate, norbornylmethyl (meth)acrylate, isobornyl (meth)acrylate, and the like.

Additional non-limiting (meth)acrylates which are suitable for preparing the copolymer powder include, many with acrylate counterparts, but are not limited to, 1,2-dimethylpropyl (meth)acrylate, 10-methylundecyl (meth)acrylate, 11-methyldodecyl (meth)acrylate, 12-methyltridecyl (meth)acrylate, 13-methyltetradecyl (meth)acrylate, 14-methylpentadecyl (meth)acrylate, 15-methylhexadecyl (meth)acrylate, 16-methylheptadecyl (meth)acrylate,2,3-epoxybutyl (meth)acrylate, 2-diethylaminoethyl (meth)acrylate, 2-ethoxyethyl (meth)acrylate, 2-ethylhexyl (meth)acrylate, 2-hydroxypropyl (meth)acrylate, 2-isocyanatoethyl (meth)acrylate, 2-methylbutyl (meth)acrylate, 3,4-epoxybutyl (meth)acrylate, 3-methylpentyl (meth)acrylate, 4,5-epoxypentyl (meth)acrylate, 4-methylpentyl (meth)acrylate, 5-methylhexyl (meth)acrylate, 6-methylheptyl (meth)acrylate, 7-methyloctyl (meth)acrylate, 8-methylnonyl (meth)acrylate, 9-methyldecyl (meth)acrylate, acetoacetoxy ethyl (meth)acrylate, butyl (meth)acrylate, cetyl (meth)acrylate, decyl (meth)acrylate, dodecyl (meth)acrylate, eicosyl (meth)acrylate, ethyl (meth)acrylate, ethylene glycol (meth)acrylate, fluorinated (meth)acrylate, glycidyl (meth)acrylate, heneicosyl (meth)acrylate, hentriacontyl (meth)acrylate, heptacosyl (meth)acrylate, heptadecyl (meth)acrylate, heptyl (meth)acrylate, hexacosyl (meth)acrylate, hexadecyl (meth)acrylate, hexadecyl (meth)acrylate, hexyl (meth)acrylate, hydroxy ethyl (meth)acrylate, hydroxy propyl (meth)acrylate, isobornyl cyclohexyl (meth)acrylate, isobornyl (meth)acrylate, isooctyl (meth)acrylate, lauryl (meth)acrylate, methoxy ethyl (meth)acrylate, methoxy polyalkylene glycol (meth)acrylate, methyl (meth)acrylate, nonacosyl (meth)acrylate, nonadecyl (meth)acrylate, nonyl (meth)acrylate, octacosyl (meth)acrylate, octadecyl (meth)acrylate, octadecyl (meth)acrylate, octyl (meth)acrylate, pentacosyl (meth)acrylate, PEG-(meth)acrylates, pentadecyl (meth)acrylate, pentaerythritol (meth)acrylate, pentyl (meth)acrylate, polyglycerin (meth)acrylate, stearyl (meth)acrylate, t-butyl aminoethyl (meth)acrylate, t-butyl (meth)acrylate, tetracosyl (meth)acrylate, tetradecyl (meth)acrylate, t-octyl (meth)acrylate, triacontyl (meth)acrylate, triazine-2,4,6-tris(2-hydroxyethyl)tris (meth)acrylate, tricosyl (meth)acrylate, tridecyl (meth)acrylate, undecyl (meth)acrylate, and other available monofunctional (meth)acrylate monomers.

Preferred homopolymer and copolymer powders are those prepared with (meth) acrylic monomers that increase the rate of dissolution, as these demonstrate an increased potential for diffusion when mixed in any of a number of monomer liquids. Such monomer inputs may include, but are not limited to, 2-ethylhexyl (meth)acrylate, dodecyl (meth)acrylate, iso-decyl (meth)acrylate, octyl (meth)acrylate, octadecyl (meth)acrylate, lauryl (meth)acrylate, hexyl (meth)acrylate, and the like.

An especially preferred monomer component of the synthesized polymer powder is 2-ethyl hexyl (meth)acrylate (2-EHMA or EHMA). EHMA has a T_{g} of −6° C., which is too low to be a suitable (meth) acrylic polymer powder by itself, but its incorporation in a copolymer enhances solubility of the polymer powder in the corresponding monomer liquid. When using a low T_{g} component, it is desirable that the powder itself or the entirety of the finished nail enhancement system does not become soft or rubbery at elevated temperatures, as may be present in tropical climates.

When synthesizing the copolymer, the weight ratio should be no more than 1-part EHMA in 2 parts of EMA. If the quantity of EHMA is more than 33%, the resulting copolymer will be undesirably soft and gum-like, and not capable of forming discrete particles. The workability characteristics of the monomer-polymer combination during application can be altered by modifying the EHMA/EMA ratio of the polymer powder, such as 1:99 up to 40:60, and combinations in between. Especially preferred are EHMA/EMA combinations of 1:99 up to 20:80.

The polymer powder may have a variety of additives, such as pigments (e.g., titanium dioxide).

The properties of the copolymer powder can be adjusted by varying the choice of monomer(s) used to synthesize the polymer or copolymer, modifying the resultant character of the polymer (e.g., the molecular weight of polymer, the ratio of chain elements in a copolymer, etc.), changing the particle size distribution of the polymer powder, and including additives with the polymer powder portion.

While EMA and 2-EHMA are used in an exemplary embodiment as co-monomers in the synthesis of the polymer powder, other combinations of monomers could be substituted in other embodiments.

For comparison, copolymer powders of EMA and lauryl (meth)acrylate, EMA and stearyl (meth)acrylate, EMA and cetyl (meth)acrylate were prepared and evaluated. Across a variety of application tests, each of these other polymer powders proved to have inferior characteristics. Inferior characteristics include lack of working (powder pickup, wet out, etc.), poor body and deposition of the ball, excess fluidity on the nail, dry time, and whether the nail enhancement resulted in a hard surface.

For comparison, a copolymer of 83% EMA and 17% n-butyl (meth)acrylate was also evaluated. When tested with a monomer liquid of the present invention, as shown in Example YY, the finished surface character of the finished nail enhancement undesirably had an uncured/tacky layer. Working characteristics, both wetting the powder and holding the shape of the monomer-polymer combination on the nail, were also poor.

For comparison, a copolymer of 91% EMA and 9%2-ethylhexyl (meth)acrylate was also evaluated. The monomer of Example YY did not pick up the powder well, the ball was slow to wet out and when placed on the nail, movement of the mixture was stiff. In this instance, it was observed that the monomer was not adequately solvating the powder. Changing the monomer liquid to one of higher EMA content however, resolved these characteristics. Such a modification of the polymer powder is hereby contemplated for use with commercial monomer liquids, those with greater than 50% EMA content.

For comparison, a copolymer of 97% EMA and 3%2-ethylhexyl (meth)acrylate was also evaluated. The high EMA monomer of Example 1 made a very wet enhancement, runny on the nail and difficult to dry. In this instance, it was observed that the monomer was solvating the powder too much. Using the same powder with the monomer of Example ZZ, the handling characteristics of the system were stiff and challenging, but the finished enhancement was very clear and free of defects.

The polymer powder part of the prior art compositions generally comprises the following ingredients: a (meth)acrylate polymer (such as poly(ethyl (meth)acrylate)) or a copolymer of methyl and ethyl (meth)acrylate, and an organic peroxide polymerization initiator (such as benzoyl peroxide). The polymer powder may have a variety of additives, such as pigments (e.g., titanium dioxide), secondary polymers (e.g., polyvinyl acetate) and flow modifiers (e.g., fumed silica). The properties of the polymer powder can be adjusted by varying the choice of monomer(s) used to synthesize the polymer or copolymer, by changing the resultant character of the polymer (e.g., the molecular weight of polymer, the ratio of chain elements in a copolymer, etc.), by changing the particle size distribution of the polymer powder, and by including additives with the polymer powder portion. Notably, the prior art describes polymer powder compositions based on the methyl (meth)acrylate and/or ethyl (meth)acrylate components.

In U.S. Pat. No. 5,738,843, the preferred powder (“polymeric filler”) is described as:

| Ingredient | Percentage |
|---|---|
| Poly(ethyl-co-methyl (meth)acrylate), | 98.88 |
| 70:30 mole ratio | |
| Benzoyl peroxide | 1.12 |

In U.S. Pat. No. 6,455,033, the powder is described as:

| Ingredient | Percentage |
|---|---|
| Poly(ethyl (meth)acrylate) | 98.4 |
| Benzoyl peroxide | 1.6 |

In U.S. Pat. No. 8,124,058, the powder composition is not itemized but describes the acrylate or (meth)acrylate polymer as being ethyl or methyl (meth)acrylate or ethyl or methyl acrylate, or a combination of one or more of these polymers.

Listed below are specific nail powders known and readily available in the market:
powder 1: polyethyl (meth)acrylate, polymethyl (meth)acrylate, benzoyl peroxide;
powder 2: polyethyl (meth)acrylate, acrylate copolymer, benzoyl peroxide;
powder 3: poly(ethyl (meth)acrylate), poly(methyl (meth)acrylate), dibenzoyl peroxide;
powder 4: polyethyl (meth)acrylate, polymethyl (meth)acrylate, dibenzoyl peroxide, silica;
powder 5: poly(ethyl and methyl (meth)acrylate), benzoyl peroxide

These embodiments demonstrate that the primary polymer powder ingredient is predictably poly ethyl (meth)acrylate, or a copolymer of ethyl (meth)acrylate and methyl (meth)acrylate, or a physical blend of poly ethyl (meth)acrylate and poly methyl (meth)acrylate, amended by an organic peroxide.

### Monomer Component

Monomer liquids commonly used in industry are based on ethyl (meth)acrylate (EMA).

In U.S. Pat. No. 5,738,843, the preferred monomer composition is described as:

| Ingredient | Percentage |
|---|---|
| Ethyl (meth)acrylate | 82.2 |
| Hydroxyethyl (meth)acrylate | 10.0 |
| Ethylene glycol di(meth)acrylate | 6.0 |
| N,N-dimethyl-p-toluidine | 0.8 |
| Tinuvin P | 1.0 |

In U.S. Pat. No. 6,455,033, the monomer composition is described as:

| Ingredient | Percentage |
|---|---|
| Ethyl (meth)acrylate | 71.0 |
| Hydroxypropyl (meth)acrylate | 10.0 |
| 2-(methacryloyloxy) ethyl acetoacetate | 6.0 |
| Tetraethylene glycol di(meth)acrylate | 12.0 |
| N,N-dimethyl-p-toluidine | 1.0 |

In U.S. Pat. No. 8,124,058, the monomer composition is described as:

| Ingredient | Percentage |
|---|---|
| Ethyl (meth)acrylate | 74.0 |
| Hydroxypropyl (meth)acrylate | 9.798 |
| Acetoacetoxy ethyl(meth)acrylate | 5.0 |
| Tetraethylene glycol di(meth)acrylate | 9.0 |
| N,N-dimethyl-p-toluidine | 1.0 |
| 2-(2′-hydroxy-5′-methylphenyl) | 0.5 |
| benzotriazole | |
| Isostearyl Isononanoate | 0.2 |
| Hydroxypropyl dimethicone | 0.2 |
| Butyrolactone | 0.2 |
| Trimethylolpropane tri(meth)acrylate | 0.1 |
| D&C violet #2 | 0.001 |
| Hydroquinone | 0.001 |

Listed below are specific acrylic nail monomer liquids known and readily available in the market:
Monomer 1: ethyl (meth)acrylate, hydroxypropyl (meth)acrylate, peg-4 di(meth)acrylate, dimethyltolylamine, drometrizole, violet 2, hydroquinone; Monomer 2: ethyl (meth)acrylate, hydroxypropyl (meth)acrylate (HPMA), dimethyltolyamine;
Monomer 3: ethyl (meth)acrylate, HEMA, glycol di(meth)acrylate, dimethyltolylamine, benzophenone-3, hydroquinone, violet 2 (CI 60725); Monomer 4: ethyl (meth)acrylate, triethylene glycol di(meth)acrylate, dimethylamylamine;
Monomer 5: ethyl (meth)acrylate, peg-4 di(meth)acrylate, HEMA, dimethyltolylamine, violet 2 (CI 60725).
   This assembly of information suggests a commercial nail monomer liquid is primarily EMA, followed by one or more functional (meth)acrylates (which serve as cross-linkers & hardeners), then associated additives. No low-odor monomer liquid examples with EMA content less than 70% could be identified.

To reduce the EMA odor, a number of (meth)acrylic monomer compositions were tested with an industry standard polymer powder (poly(ethyl (meth)acrylate)). Table 1 demonstrates that all tested monomers resulted in an inadequate finished nail surface:

TABLE 1

| Monomer Liquid Test Composition | | Polymer Powder | |
|---|---|---|---|
| 56% of primary monomer shown below | | poly(ethyl (meth)acrylate) | |
| 41% EMA initiator | | Unpolymerized | |
| | Surface Tack | Surface | |
| Primary Monomer | @ 5 Minutes | @ 10 Minutes | Comments |
| Hydroxyethyl (meth)acrylate | 2 | 2 | skin irritant & |
| | | | yellow |
| Hydroxybutyl (meth)acrylate | 4 | 2 | |
| Iso-Butyl (meth)acrylate | 4 | 3 | strong odor |
| n-Butyl (meth)acrylate | 5 | 5 | strong odor |
| Triethylene Glycol | 1 | 2 | |
| di(meth)acrylate | | | |
| PEG 200 di(meth)acrylate | 4 | 3 | |
| 1,4 Butanediol | 3 | 2 | |
| di(meth)acrylate | | | |
| 1,3-butanediol | 3 | 3 | |
| di(meth)acrylate | | | |
| Glycerol di(meth)acrylate | 3 | 3 | yellow |
| Trimethylolpropane | 2 | 2 | |
| tri(meth)acrylate | | | |
| 1,6-hexanediol | 4 | 3 | |
| di(meth)acrylate | | | |
| 2-Ethylhexyl (meth)acrylate | 5 | 5 | |
| Ethylene glycol | 3 | 2 | |
| di(meth)acrylate | | | |
| Hydroxypropyl (meth)acrylate | 4 | 2 | |
| t-Butyl (meth)acrylate | 5 | 5 | strong odor |

Ranking of 1-5, with 1 being best and equivalent to Example 1

Applying the same evaluation, the following prior art compositions resulted in runny, non-drying systems as shown in Table 2:

TABLE 2

| | | Unpolymerized | |
|---|---|---|---|
| | Surface Tack | Surface | |
| Primary Monomer | @ 5 Minutes | @ 10 Minutes | Comments |
| Example 3 (U.S. Pat. No. | 4 | 3 | Very fluid |
| 4,871,534, example IV) | | | |
| Example 4 (U.S. Pat. No. | 3 | 4 | Very fluid |
| 5,098,696, example II) | | | |

Polymer powder is poly(ethyl (meth)acrylate)
Ranking of 1-5, with 1 being best and equivalent to Example 1

The same experiments were conducted using the following exemplary copolymers of the present invention, with the results shown in Table 3:

TABLE 3

| | | Polymer Powder | |
|---|---|---|---|
| Monomer Liquid Test Composition | | Copolymer of the instant | |
| 56% of primary monomer shown below | | invention (Example ZZ) | |
| 41% EMA - Initiator | | Unpolymerized | |
| | | Surface Tack | Surface |
| Primary Monomer | @ 5 Minutes | @ 10 Minutes | Comments |
| Hydroxyethyl (meth)acrylate | 1 | 1 | skin irritant & |
| | | | yellow |
| Hydroxybutyl (meth)acrylate | 3 | 3 | |
| iso Butyl (meth)acrylate | 3 | 3 | strong odor |
| n-Butyl (meth)acrylate | 1 | 1 | strong odor |
| Triethylene Glycol | 2 | 1 | |
| di(meth)acrylate | | | |
| PEG 200 di(meth)acrylate | 2 | 2 | |
| 1,4 Butanediol di(meth)acrylate | 1 | 1 | |
| 1,3-butanediol di(meth)acrylate | 1 | 1 | |
| Glycerol di(meth)acrylate | 2 | 1 | yellow |
| Trimethylolpropane | 4 | 2 | |
| tri(meth)acrylate | | | |
| 1,6-hexanediol di(meth)acrylate | 2 | 2 | |
| 2-Ethylhexyl (meth)acrylate | 2 | 4 | |
| Ethylene Glycol di(meth)acrylate | 1 | 1 | |
| Hydroxypropyl (meth)acrylate | 1 | 1 | |
| t-Butyl (meth)acrylate | 1 | 1 | strong odor |

Ranking of 1-5, with 1 being best and equivalent to Example 1

Preferred embodiments of the prior art were tested using the improved polymer powders of the instant invention. The working characteristics improved, but were still deficient compared to expectations as shown in Table 4:

TABLE 4

| | | Unpolymerized | |
|---|---|---|---|
| | Surface Tack | Surface | |
| Primary Monomer | @ 5 Minutes | @ 10 Minutes | Comments |
| Example 3 (U.S. Pat. No. | 2 | 3 | Very fluid |
| 4,871,534, example IV) | | | |
| Example 4 (U.S. Pat. No. | 2 | 3 | Very fluid |
| 5,098,696, example II) | | | |

Polymer powder is Example ZZ of the present invention
Ranking of 1-5, with 1 being best and equivalent to Example 1

It is observed that the functional performance of many monomer liquid combinations can be dramatically improved, simply by changing the composition of the synthesized polymer powder. From this evaluation, assembly of a preferred monomer liquid can be accomplished.

In an exemplary embodiment, the monomer component is comprised of the following ingredients, based upon the total weight of monomer liquid:
a) from 1 to 90%, such as 1 to 70%, such as 5 to 70%, such as 10 to 70%, such as 20 to 70%, such as 25 to 70%, such as 30 to 70%, such as 40 to 70% of a di-, tri-, or multi-functional (meth)acrylic ester, and
b) from 10 to 99%, such as 30 to 99%, such as 30 to 95%, such as 30 to 90%, such as 30 to 80%, such as 30 to 75%, such as 30 to 70%, such as 30 to 60% of a (meth)acrylate ester with an alkyl side chain of 3 or less carbon atoms, and
c) an accelerator, typically present in the monomer component in an amount of between 0 and up to 5%, such as 0.05 to 5%, such as 0.1 to 5%, such as 0.05 to 3%, such as 0.1 to 3%.

In addition to the above components, the monomer component may optionally contain a polymerization inhibitor, such as, but not limited to, butylated hydroxytoluene (BHT), hydroquinone (HQ), hydroxytoluene 4-tert-butylcatechol (TBC), 2-tert-Butyl-4,6-dimethylphenol (TOPANOL A), N,N-diethylhydroxylamine, 4-methoxyphenol (MEHQ), phenothiazine, 2,6-di-tert-butyl-p-cresol, 2-tert-butyl-1,4-benzoquinone, 1,4-benzoquinone, tert-butylhydroquinone, 6-tert-butyl-2,4-xylenol, or 2,6-di-tert-butylphenol)) to prevent premature reaction of the (meth)acrylate monomers and to assure adequate shelf life.

Also, suitable light stabilizers include any number of which are well known in the art, such as, but not limited to, benzotriazole (BTZ), hindered amine light stabilizers (HALS), triazine (HPT) UV absorbers and other free radical scavengers, examples of which may include, 2-(2-hydroxy-5-methylphenyl)benzotriazole, bis(1,2,2,6,6-pentamethyl-4-piperidyl) sebacate, 2-hydroxyphenyl-s-triazine, 85% in 1-methoxypropan-2-ol, 2-hydroxyphenyl-s-triazine, hydroxyphenyl-triazine, methanone, [2-hydroxy-4-(octyloxy)phenyl]phenyl-, methanone, bis(2-hydroxy-4-methoxyphenyl)-, dimethyl (p-methoxybenzylidene)malonate, benzenesulfonic acid, and 3,3′-carbonylbis[4-hydroxy-6-methoxy-, sodium salt (1:2)), may optionally be added in the monomer liquid portion to prevent light-activated polymerization and for the finished nail enhancement, to resist yellowing due to ultraviolet light.

Auxiliary components such as dyes (such as, but not limited to, D&C Violet dye (#2), D&C Green dye (#5, 6), D&C Orange dye (#4), D&C Red dye (#17, 21, 22, 27, 28, 33), D&C Yellow dye (#8, 10, 11)) may be included so as to modify color post-cure properties.

In an exemplary embodiment, one or more low-odor (meth)acrylates described herein make up a majority of the formulated monomer liquid, preferably greater than 50%, such as greater than 60%, such as greater than 70%, such as greater than 80%, such as greater than 90% by weight, of the total monomer component.

In an exemplary embodiment, one or more di-, tri-, tetra- and/or multi-functional (meth)acrylates are the primary ingredient(s) in the monomer component. Such functional (meth)acrylates serve to increase the mechanical strength of the cured polymer nail/coating, improving properties such as stiffness, tensile strength, abrasion resistance, and chemical resistance. The (meth)acrylate monomer compositions of the monomer component are unique in their combination of providing high solvency, low volatility, low toxicity, and reduced odor, particularly when used in combination with the polymer component of the invention as described herein.

To obtain the desired cured polymer properties within this particular system, the selection of and formulation of these functional (meth)acrylates is necessary. Many functional (meth)acrylate or (meth)acrylate monomer combinations are well known in the art. Suitable candidates for the monomer(s) in the monomer component include, but are not limited to, polyethylene glycol di(meth)acrylate, 1,6-hexanediol di(meth)acrylate, 1,9-nonanediol di(meth)acrylate, 1,4-butanediol di(meth)acrylate, 1,3-butanediol di(meth)acrylate, 1,12-dodecandiol di(meth)acrylate, 1,10-decanediol di(meth)acrylate, diurethane di(meth)acrylate, ethylene glycol di(meth)acrylate, triethylene glycol di(meth)acrylate, tetraethylene glycol di(meth)acrylate, 1,5-pentanediol di(meth)acrylate, 1,4-phenylene diacrylate, pentaerythritol (meth)acrylate, polyethylene glycol di(meth)acrylate, diethylene glycol di(meth)acrylate, pentaerythritol di(meth)acrylate, trimethylolpropane tri(meth)acrylate, propoxylated trimethylolpropane triacrylate, propoxylated glycerol triacrylate, trimethylolpropane (EO) tri(meth)acrylate, tricylcodecanol (meth)acrylate, glycerol tri(meth)acrylate, pentaerythritol tri(meth)acrylate, ethoxylated glycerine trimethacylate, 2,2-bis[4-(2′-hydroxy-3′-methacryloyloxy-propoxy)phenyl]propane, 1,1,1-Tris[4-(2′-hydroxy-3′-methacryloyloxypropoxy)phenyl]ethane, and or tris [4-(2′-hydroxy-3′ methacryloyloxypropoxy)phenyl]methane. Many available ethoxylated, propoxylated, difunctional and trifunctional monomers are essentially identical, but for chain length, number of repeating units. Examples of such include, but are not limited to, polyethyleneglycol (di) (meth)acrylate, i.e., PEGDMA 200, 400, 600, 1000, polypropyleneglycol (di) (meth)acrylate, poly(ethylene, propylene, tetraethylene, butylene) glycol (meth)acrylate, methoxy (octoxy, lauroxy, stearoxy, phenoxy, etc.) -polyethyleneglycol-(meth)acrylate or -polyethyelene-polypropyleneglycol-(meth)acrylate. Further, any of these embodiments possessing an acrylate functionality in place of the (meth)acrylate moiety are also suitable.

In an exemplary embodiment, one or more of those functional (meth)acrylate monomers having little, or no odor are included in the monomer component of the present invention. In an exemplary embodiment, one or more of those functional (meth)acrylate monomers with a glass transition temperature greater than 20° C. (such as greater than 30° C., such as greater than 40° C., such as greater than 50° C.) are included in the monomer component of the present invention. In an exemplary embodiment, one or more of those functional (meth)acrylate monomers that can chemically cure with a resultant coating surface free or substantially free of an un-polymerized/oxygen inhibition surface layer are included in the monomer component of the present invention. In an exemplary embodiment, one or more of those functional (meth)acrylate monomers that can chemically cure in no less than 60 seconds and no more than 600 seconds (such as no less than 150 seconds and no more than 500 seconds, such as no less than 200 seconds and no more than 400 seconds) are included in the monomer liquid of the present invention. Such functionalized (meth)acrylate monomers generally are used in an amount from about 1 percent to about 70 percent, such as 5 to 70 percent, such as 5 to 60 percent, such as 10 to 60 percent, such as 15 to 60 percent, such as 10 to 50 percent, by weight of monomer liquid.

Due to the strength of their intermolecular forces, different substances have different vapor pressures at a given temperature. For molecules to escape into the vapor phase, they must have sufficient kinetic energy to overcome the intermolecular forces of other molecules at the surface of the liquid. It follows, therefore, that substances with weaker intermolecular forces will allow more molecules to escape into the gas phase at equilibrium—in other words, they will have a higher vapor pressure. Substances with strong intermolecular forces will have a lower vapor pressure because fewer molecules will have sufficient kinetic energy to escape at a given temperature.

Substances with high vapor pressures more readily change into a vapor state. Many small organic compounds are volatile, including most scent compounds. Low vapor pressure is not the only measure of smell, but low vapor pressure monomers will have a reduced availability of vapor, regardless of other odor aspects, such as intensity or favorability.

A number of functional (meth)acrylate monomers satisfy these screening criteria. Non-limiting examples include triethylene glycol di(meth)acrylate (TEGDMA), diethylene glycol di(meth)acrylate, tetraethylene glycol di(meth)acrylate, trimethylolpropane tri(meth)acrylate (TMPTMA), and PEG 200 di(meth)acrylate. Such functional (meth)acrylate monomers were evaluated and found to have various deficiencies, of working character, color, surface curing and/or finished nail aesthetics.

Preferred functional (meth)acrylate monomers include glycerol di(meth)acrylate (GDMA), or 1,4-butanediol di(meth)acrylate (tetraethylene (meth)acrylate or BDDMA), or 1,3-butanediol di(meth)acrylate), or 4-hydroxypropyl (meth)acrylate (HPMA), or 4-hydroxybutyl (meth)acrylate (HBMA), or 1,6-hexanediol di(meth)acrylate), or ethylene glycol di(meth)acrylate (EGDMA), or combinations thereof. The most preferred functional (meth)acrylate monomer is 1,4-butanediol di(meth)acrylate.

Another constituent in the monomer liquid may contribute solvency to the reaction with the polymer powder and create a hard finished nail enhancement. One example is a high T_{g} monomer such as EMA. As described herein, EMA has a high vapor pressure and a negative (acrid) smell. Use of EMA should preferably be limited to no more than 50% of the low-odor monomer combination, or less.

Cross-linking (meth)acrylic monomers are known in industry to improve coating hardness. These materials provide additional reaction sites, increasing density during formation of polymer architecture.

The molecular structure between the end (meth)acrylic groups is termed a “spacer”, the size and configuration of the spacer helps to determine the physical and mechanical properties of the resulting polymer structure. Depending on a number of formulation, application and use characteristics, brittle tendencies may also result. Brittleness is thought to be a result of the (meth)acrylic chains being chemically bound, preventing deformation when impacted.

With an acrylic nail enhancement, brittleness is embodied by free edge chipping, a failure of the enhancement in the area beyond the end of the fingertip. Among other approaches, brittleness of an acrylic nail enhancement can be reduced with any number of known additives. Such additives include rubber, core shell acrylic impact modifiers, glycol esters, polyesters (including acetate esters, alcohol esters, etc.), phthalates, benzoates, surfactants, and silicones. The formulation challenge with any is assuring that the plasticization does not create an undesirable side effect, such as an incompatible film or haze rising to the surface of the nail enhancement, as may result from a non-polymerized domain.

In some instances, reduction of brittleness can be accomplished simply by changing the (meth)acrylate spacer. For instance, a longer chain mono(meth)acrylate or di(meth)acrylate may serve to interrupt the polymer crystallinity, thereby eliminating chipping and improving wear for the user. Examples of longer chain mono(meth)acrylates might include, without limitation, decyl (meth)acrylate, lauryl (meth)acrylate, myristyl (meth)acrylate, cetyl (meth)acrylate, stearyl (meth)acrylate, behenyl (meth)acrylate and the like. Di(meth)acrylates with larger spacers may include, but are not limited to, 1,6-hexanediol di(meth)acrylate, neopentyl glycol di(meth)acrylate, diethylene glycol di(meth)acrylate, triethylene glycol di(meth)acrylate, polyethylene glycol di(meth)acrylate, and the like.

Amine accelerators are generally known in the art and include aromatic tertiary amines such as N,N-dimethyl-p-toluidine (DMPT), N,N-dihydroxyethyl-p-toluidine, N,N-dimethyl aniline, 1-phenylpyrrolidine, and the like. A preferred amine accelerator is an ethoxylated p-toluidine, such as a reaction mass of 2,2′-[(4-methylphenyl)imino] bisethanol and ethanol 2-[[2-(2-hydroxyethoxy)ethyl](4-methylphenyl)amino]-. Also preferred is N-methyl-N-(2-hydroxyethyl)-p-toluidine (MHPT) and derivatives thereof, such as but not limited to, N-methyl-N-(2-hydroxypropyl)-p-toluidine (2HMPT). Formulations using DMPT were observed to have an undesirable un-polymerized/oxygen inhibition layer, while those using MHPT or ethoxylated p-toluidine did not.

The monomer liquid may optionally contain auxiliary components such as dyes, polymerization inhibitors such as BHT and/or MEHQ, ultraviolet light absorbers and fragrances.

### Vapor Pressure

Vapor pressure of the monomer liquid compositions can be calculated using Raoult's Law. This value indicates neither the intensity of the vapor nor a given individual's preference for the odor. Non-limiting examples of vapor pressures for common (meth) acrylic nail monomer liquids include those listed in Table 5:

TABLE 5

| | Calculated |
|---|---|
| | Vapor Pressure |
| Reference | (mmHg) |
| Example 1, conventional monomer sold in | 17.7 |
| industry, as shown in Table 11 | |
| Example ZZ, A preferred monomer liquid of the | 9.6 |
| invention | |
| Example 4, (U.S. Pat. No. 5,098,696, Example II) | 4.0 |
| Example 3, (U.S. Pat. No. 4,871,534, Example IV) | 2.0 |
| Example 2, “Low-odor” monomer sold in | 0.2 |
| industry | |

Analytically, the monomer liquid vapor pressure of the present invention is determined to be approximately 50% less than commercially available monomers, while also providing enhanced use character.

### Odor

To further examine the nature of the invention, various monomer liquids were analyzed by Odor Science & Engineering, Inc., 105 Filley Street, Bloomfield, CT. The testing was conducted using the “headspace” test method as described below.

Headspace Odor Evaluation Procedure-A standardized volume (10 ml aliquot) of each sample was injected into Tedlar gas sampling bags pre-filled with 20 liters of carbon-filtered odor-free air. The bags were maintained at 70□ F for a 2-hour period. The bags were gently mixed during the 2-hour period to ensure a saturated headspace vapor concentration was created. After the two hours, a measured amount of the headspace air within each sample bag was transferred into a new 12-liter Tedlar bag for sensory evaluation.

The headspace samples were analyzed by dynamic dilution olfactometry using a trained and screened odor panel. The odor panelists were chosen from a pool of panelists who actively participate in ongoing olfactory research and represent an average to above average sensitivity when compared to a large population. The samples were quantified in terms of dilution-to-threshold (D/T) ratio and odor intensity in accordance with ASTM Methods E-679-04 with the results shown in Table 6:

TABLE 6

| | Odor | | |
|---|---|---|---|
| | Concentration | | Hedonic |
| Sample ID | D/T ⁽¹⁾ | Intensity ⁽²⁾ | Tone ⁽³⁾ |
| Commercial monomer | 211,210 | 2.0 | −4.2 |
| liquid of Example 1 | | | |
| Monomer liquid of | 26,606 | 2.0 | −3.5 |
| Example ZZ, Present | | | |
| Invention | | | |
| Monomer of Example 4, | 8,313 | 2.0 | −3.5 |
| U.S. Pat. No. 5,098,696, | | | |
| Preferred embodiment | | | |

1. D/T=dilutions-to-threshold (n=8): ASTM E-679-04
2. Odor panel intensity rating: Intensity=0-8 based on the n-butanol intensity reference scale: ASTM Method E-544-10
3. Odor panel avg. hedonic tone rating at intensity level of 2.0, where hedonic tone=degree of pleasantness/unpleasantness (−8=extremely unpleasant, 0=neutral, +8=extremely pleasant)

The monomer liquid odor concentration of the claimed invention was found to be 87% lower than the commercial monomer liquid standard. The six odor panelists were also asked to rate the hedonic tone (degree of pleasantness or unpleasantness) of the samples at varying dilution levels. When the diluted odor sample became detectable and reached an intensity level of 2.0 (on the 0-8 point butanol intensity scale-ASTM E-544-10), the panelists were asked to rate the degree of pleasantness (or unpleasantness) of the odor. The hedonic tone was measured by means of a 17-point category estimate scale ranging from +8 (extremely pleasant), +6 (very pleasant), +4 (moderately pleasant), +2 (slightly pleasant) 0=neutral, −2 (slightly unpleasant), −4 (moderately unpleasant), −6 (very unpleasant) and −8 (extremely unpleasant). All of these equalized samples were judged by the panelists to have a “moderately” unpleasant odor at an intensity level of 2.0 with hedonic ratings ranging between −3.5 and −4.0.

### Color

To quantify the color improvement, comparisons were prepared and tested on a X-Rite benchtop sphere spectrophotometer, per the requirement of ASTM E1164, Standard Practice for Obtaining Spectrometric Data for Object-Color Evaluation.

In precise color communication, colors are expressed numerically. The color numbers are coordinates of a 3-dimensional color space. In design and manufacturing, the CIE L*a*b* and L*C*h° Color spaces are used to ensure product compliance.

CIE L*C*h° Color space is a vector representation of the CIE 1976 L*a*b* color space. With the LCH color model, L* indicates lightness-lighter or darker, C* represents the chroma axis and h° is the hue angle.

Discs of the representative systems were prepared, uniformly measured 1.5 inches diameter and a depth of 3/16 of one inch. Each was measured on an X-Rite sphere benchtop spectrophotometer, with the output plotted against a CIE L*C*h° color space model. The color measurement results of the acrylic nail systems are shown in Table 7:

TABLE 7

| Description of acrylic nail system | L* | C* | h° |
|---|---|---|---|
| Example 1, Well known | 83.96 | 1.37 | 44.62 |
| monomer and polymer | | | |
| Example 2, Well known | 65.72 | 19.17 | 78.09 |
| low-odor monomer and polymer | | | |
| Example 3, (U.S. Pat. No. | 79.61 | 8.15 | 88.24 |
| 4,871,534, example IV) | | | |
| Example 4, (U.S. Pat. No. | 74.65 | 15.12 | 88.31 |
| 5,098,696, example II) | | | |
| Example ZZ, A preferred | 83.76 | 7.10 | 91.34 |
| embodiment of the invention | | | |

Analytically, the instant invention has a brightness greater than results from earlier inventions. Further, the instant invention has a brightness equal to the conventional monomer and polymer systems.

### Haze

The improved visual presentation of the present invention is quantified by measuring transmission haze. Transmission haze is defined as the percentage of incident light scattering as it passes through a transparent material, resulting in poor visibility and/or glare. There are several factors responsible for light scattering in the combined and cured monomer liquid and polymer powder, including impurities contained in the plastic material, surface roughness, and/or internal optical irregularities caused by crystallization or the material's level of crystallinity. Other factors include inhomogeneities (due to density differences, the presence of fillers, pigments, etc.), porosity, crystal size structure (such as may result from crosslinking), degree of mechanical and chemical degradation, and/or environmental factors (such as weathering or surface abrasion).

The standard for transmission haze measurement is ASTM D-1003. This test method, “Standard Test Method for Haze and Luminous Transmittance of Transparent Plastics”, covers the evaluation of specific light-transmitting and wide-angle-light-scattering properties of planar sections of essentially transparent plastic. As described in Procedure B of ASTM D-1003, the light is uniformly distributed by a matte white highly reflective coating on the sphere walls and measured by a photodetector positioned at 90° from the entrance port. A baffle mounted between the photodetector and the entrance port prevents direct exposure from the port. The exit port immediately opposite the entrance port contains a light trap to absorb all light from the light source when no sample is present. A shutter in this exit port coated with the same coating as the sphere walls allows the port to be opened and closed as required. Transmittance haze is measured with the exit port open.

Samples were prepared of comparative systems, uniformly measured at 1.5 inches diameter and a depth of 3/16 inch. Each was measured on a DataColor benchtop sphere spectrophotometer, with the following results reported in SI units (candela) as shown in Table 8:

TABLE 8

| | Monomer | | |
|---|---|---|---|
| Description | Liquid | Polymer Powder | Haze |
| Conventional monomer | Example 1 | Example 1 | 41.8 |
| and polymer | | | |
| Conventional monomer | Example 1 | Poly(ethyl-co-methyl | 65.4 |
| and polymer | | (meth)acrylate) | |
| Low-odor monomer | Example 2 | Example 2 | 98.8 |
| and polymer | | | |
| established in industry | | | |
| U.S. Pat. No. 4,871,534, | Example 3 | Example 3 | 85.6 |
| Example IV | | | |
| U.S. Pat. No. 5,098,696, | Example 4 | Example 4 | 94.1 |
| Example II | | | |
| Preferred composition | Example ZZ | Example ZZ | 38.7 |
| 1 | | | |
| Preferred composition | Example 1 | Example ZZ | 24.4 |
| 2 | | | |

The lower the haze measurement value, the higher the clarity. In industry, existing low-odor products are solely monomer liquids. Whether the monomer liquid is low-odor or not, as practiced in industry, the same polymer powders are used. Compared to both market standard low-odor and conventional acrylic nail combinations, the present invention exhibits significantly higher clarity.

### Photos

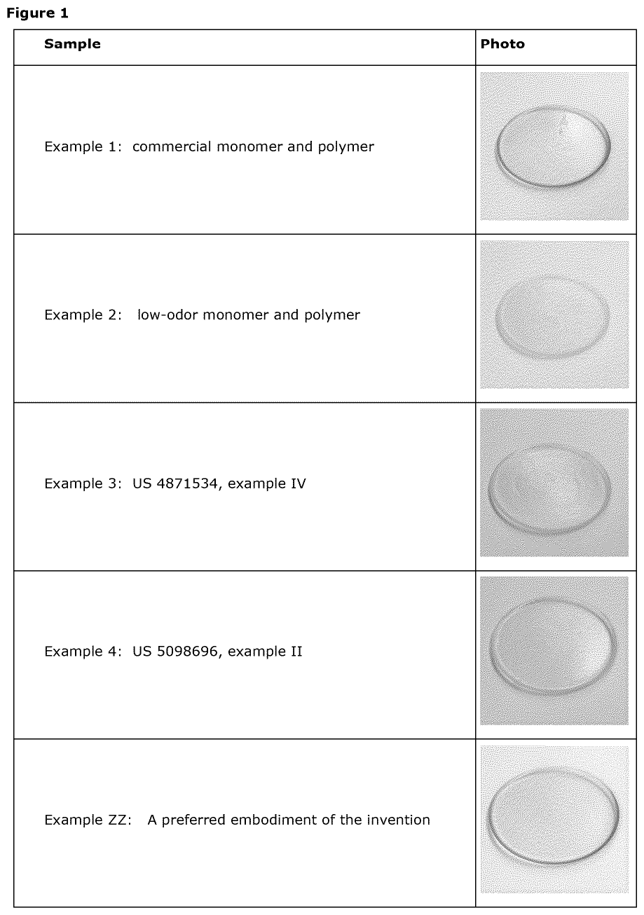

The cured and finished nail enhancement resulting from known low-odor systems are typically darker, yellower and and/or more opaque than preferred. By comparison, the monomer-polymer mixture of the present invention results in a clear application. FIG. 1 depicts photographs of various samples of the prior art, compared to an embodiment of the present invention.

### Glass Transition Temperature

Nail enhancements are employed under a variety of environmental conditions. In some regions of the world, ambient temperatures can readily exceed 100° F. (38° C.), 105° F. (40° C.), or even 110° F. (43° C.). As a result, nail coatings must be able to withstand these conditions without undergoing a physical change.

Glass transition temperature (T_{g}) is a measure of coating properties. If ambient temperatures approach the T_{g} of the nail enhancement, the coating properties can change, becoming hazy, tacky, etc. With such a fashion item as nail enhancements, deterioration of their condition would create a negative use experience.

To demonstrate thermal properties of the finished enhancement, selected monomer liquids and polymer powders have been combined to form nail tips, with the results shown in Table 9:

TABLE 9

| Monomer Liquid | Polymer Powder | T_{g} , 1^{st} Heat, ° C. |
|---|---|---|
| Example 1 | Example 1 | 52 |
| Example 2 | Example 2 | 48 |
| Example 3 | Example 3 | 47 |
| Example 4 | Example 4 | 44 |
| Example ZZ | Example ZZ | 51 |

The T_{g} values for the finished nail tips obtained from the present invention are the same as that of Example 1, the control. The first heat T_{g} for U.S. Pat. No. 4,871,534, example IV is 5° C. below the control. The first heat T_{g} for U.S. Pat. No. 5,098,696, example II, is 8° C. less than the control.

### Surface Hardness

Shore Hardness is a measure of the resistance a material has to indentation. Hardness of the below samples were measured using a DeFelsko PosiTector® SHD Tester. This device conforms to ASTM D-2240, “Standard Test Method for Rubber Property-Durometer Hardness”. The formed nail tips were measured on the “D” scale, “D” scale being for hard rubbers, semi rigid plastics and hard plastics.

Samples were prepared of comparative systems, uniformly measured at 1.5 inches diameter and a depth of 3/16 inch. Tests were conducted after 10 and 60 minutes. The tester was recalibrated before and after every test sequence. Results are shown in Table 10:

TABLE 10

| | Shore “D” | Shore “D” |
|---|---|---|
| | Hardness | Hardness |
| Description of acrylic nail system | @ 10 minutes | @ 60 minutes |
| Example 1, monomer and polymer | 57.8 | 68.3 |
| Example 2, low-odor monomer | 54.8 | 62.0 |
| and polymer | | |
| Example 3, (U.S. Pat. No. | 45.8 | 62.0 |
| 4,871,534, example IV) | | |
| Example 4, (U.S. Pat. No. | 56.0 | 67.5 |
| 5,098,696, example II) | | |
| Example ZZ, A preferred embodiment | 65.1 | 77.6 |
| of the invention | | |

N = 6

On the scale of 0-100, the instant invention demonstrates superior surface hardness upon 10-minute cure. This is a critical characteristic for a 2-part acrylic nail enhancement system as used in a nail salon. Low-odor systems of the prior art eventually attain an acceptable hardness, but the extended time required to do so is impractical in use.

### EXAMPLES

The following illustrate compositions that have found particular utility in the preparation of preferred artificial nail enhancements.

Example 1 represents a commercial monomer and polymer acrylic nail system.

| Monomer Liquid | | Polymer Powder | |
|---|---|---|---|
| Ethyl (meth)acrylate | 80% | Poly(ethyl | 99% |
| | | (meth)acrylate) | |
| Tetraethylene glycol | 19% | Benzoyl peroxide | 1% |
| di(meth)acrylate | | | |
| Dimethyl para-toluidine | 1% | | |

Example 1 demonstrates superior handling and represents a preferred nail enhancement. However, the odor of the monomer liquid is strong and acrid.

Example 2 represents a commercial low-odor monomer and polymer acrylic nail system.

| Monomer Liquid | | Polymer Powder | |
|---|---|---|---|
| Hydroxyethyl (meth)acrylate | 79% | Poly(ethyl | 99% |
| | | (meth)acrylate) | |
| Tetraethylene glycol | 20% | Benzoyl peroxide | 1% |
| di(meth)acrylate | | | |
| Dimethyl para toluidine | 1% | | |

Example 2 does not pick up the powder well, nor does the monomer wet the powder. The ball also does not release effectively from the brush and once applied, the monomer-polymer mixture undesirably puddles.

Example 3 represents U.S. Pat. No. 4,871,534, example IV.

| Monomer Liquid | | Polymer Powder | |
|---|---|---|---|
| Methoxyethoxyethyl | 48% | Poly(ethyl | 99% |
| (meth)acrylate | | (meth)acrylate) | |
| Ethoxyethoxyethyl (meth)acrylate | 18% | Benzoyl peroxide | 1% |
| Diethylene glycol di(meth)acrylate | 34% | | |
| Dimethyl para-toluidine | 1% | | |

Example 3 exhibits many poor attributes. It does not pick up the powder well, the ball does not wet out, nor does the monomer-polymer mixture release easily from the brush. When applied, the mixture is very fluid and cannot be shaped.

Example 4 represents U.S. Pat. No. 5,098,696, example II.

| Monomer Liquid | | Polymer Powder | |
|---|---|---|---|
| Methoxyethoxyethyl | 40% | Poly(ethyl | 99% |
| (meth)acrylate | | (meth)acrylate) | |
| Ethylene glycol di(meth)acrylate | 28% | Benzoyl peroxide | 1% |
| Hydroxyethyl (meth)acrylate | 31% | | |
| Dimethyl para toluidine | 1% | | |

Example 4 demonstrates some improvement over Example 3. The monomer-dipped brush picks up the powder better, although the wet out of the ball is still mediocre. Sculpting of the ball is difficult.

Example 5 represents U.S. Pat. No. 5,603,924, preferred embodiment.

| Monomer Liquid | | Polymer Powder | |
|---|---|---|---|
| Ethyl (meth)acrylate | 82% | Poly(ethyl-co-methyl | 99% |
| | | (meth)acrylate) | |
| Hydroxyethyl (meth)acrylate | 10% | Benzoyl peroxide | 1% |
| Ethylene glycol di(meth)acrylate | 6% | | |
| Dimethyl para toluidine | 1% | | |

Example 5 is equivalent in performance to Example 4. Once applied on the nail, the ball is liquid, thus preventing effective sculpting. The liquid smells strongly of EMA.

Example YY compares the use of n-butyl (meth)acrylate as an ingredient in the polymer powder.

| Monomer Liquid | | Polymer Powder | |
|---|---|---|---|
| 1,4 Butanediol di(meth)acrylate | 67% | Poly(ethyl-co-n-butyl) | 99% |
| | | (meth)acrylate | |
| Ethyl (meth)acrylate | 32% | Benzoyl peroxide | 1% |
| Ethoxylated para toluidine | 1% | | |

Example YY exhibited partial surface tack after curing. The characteristics of pickup, wet out, and holding of shape on the nail all needed improvement.

Example ZZ represents a preferred embodiment of the present low-odor invention.

| Monomer Liquid | | Polymer Powder | |
|---|---|---|---|
| 1,4 Butanediol | 67% | Poly(ethyl-co-2-ethylhexyl) | 99% |
| di(meth)acrylate | | (meth)acrylate | |
| Ethyl (meth)acrylate | 32% | Benzoyl peroxide | 1% |
| N-methyl-N-(2- | 1% | | |
| hydroxyethyl)-p-toluidine | | | |

Example ZZ is functionally equivalent to Example 1, with an ideal combination of preferred pickup, wet out, removal from the brush, sculpting, and holding of the shape of the monomer-polymer slurry on the nail. The slurry was observed to cure in a satisfactory manner and exhibited a favorable odor aesthetic. Buffing the enhancement resulted in a fine dust, a characteristic that is preferred in the salon industry. In an exemplary embodiment, Example ZZ further contains one or more of a pigment, fragrance and UV absorber. In another embodiment, the amount of the initiator present in the monomer liquid component may be modified as needed to achieve a desired curing time. In yet another embodiment, the amount of the benzoyl peroxide may be varied as desired to adjust setting speed.

The following studies relate to unexpected benefits associated with variations in particle size of the polymer powder component. Commercial monomer liquids were combined with respective polymer powders, formed into a nail tip, and evaluated.

Depending on the manufactured composition, application of the acrylic nail enhancement may vary. The objective of the nail enhancements is to improve the problem of visual defects while also delivering a beneficial set of working characteristics when using conventional higher EMA monomer liquids.

In all experiments, the monomer liquid was the commercial recipe shown in Table 11.

TABLE 11

| Ingredient | Percentage |
|---|---|
| Ethyl (meth)acrylate | 80% |
| Tetraethylene glycol | 19% |
| di(meth)acrylate | |
| Dimethyl para-toluidine | 1% |

### Working Characteristics

The individual combinations described below were rated by a team of experienced nail industry professionals. Rating criteria for working characteristics included pick-up, wet out, transfer and shaping.

In the market, acrylic nail powders typically have a Mv particle size near 60 microns. Testing copolymer ratios of this approximate particle size generated the results shown in Table 12:

TABLE 12
Working characteristics using common particle size powder
Rating scale of 1-5, 1 being best/5 being worst

| | | | Rating of |
|---|---|---|---|
| EMA % in | EHMA % in | | Working |
| Powder | Powder | Mv Particle Size | Characteristics |
| 83 | 17 | 51 | 4.00 |
| 85 | 15 | 51 | 3.00 |
| 88 | 12 | 58 | 2.00 |
| 90 | 10 | 63 | 2.25 |
| 93 | 7 | 54 | 1.25 |

It is observed that increasing the Mv particle size of the copolymer ratios Improved the working character as shown in Table 13:

TABLE 13
Working characteristics using larger particle size powder
Rating scale of 1-5, 1 being best/5 being worst

| | | | Rating of |
|---|---|---|---|
| EMA % in | EHMA % in | | Working |
| Powder | Powder | Mv Particle Size | Characteristics |
| 83 | 17 | 77 | 1.75 |
| 85 | 15 | 75 | 3.00 |
| 88 | 12 | 81 | 1.15 |
| 90 | 10 | 76 | 1.00 |
| 93 | 7 | 80 | 1.00 |

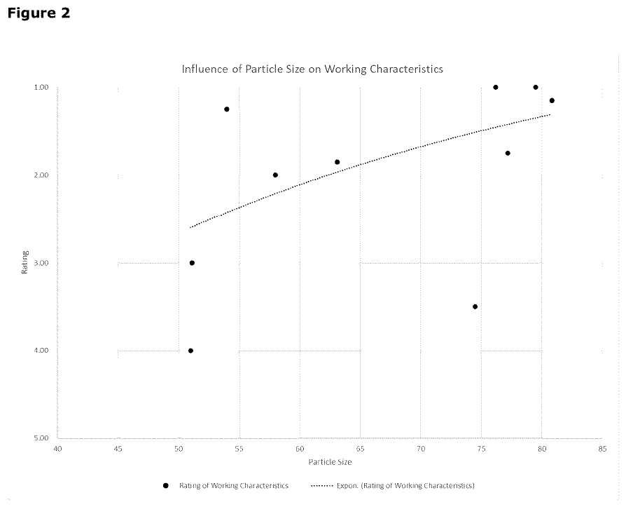

By consolidating and charting the data in Table 12 and Table 13, as shown in FIG. 2, the working characteristics of the powder appear to be improved with a particle size greater than commonly used in industry.

Table 14 uses the data in Table 12 and Table 13, rearranging the results to illustrate the relationship between working characteristics and the EMA/EHMA ratio:

TABLE 14
Working characteristics from varying copolymer ratios
Rating scale of 1-5, 1 being best/5 being worst

| | | | Rating of |
|---|---|---|---|
| EMA % in | EHMA % in | | Working |
| Powder | Powder | Mv Particle Size | Characteristics |
| 90 | 10 | 76 | 1.00 |
| 93 | 7 | 80 | 1.00 |
| 88 | 12 | 81 | 1.15 |
| 93 | 7 | 54 | 1.25 |
| 83 | 17 | 77 | 1.75 |
| 88 | 12 | 58 | 2.00 |
| 90 | 10 | 63 | 2.25 |
| 85 | 15 | 75 | 3.00 |
| 85 | 15 | 51 | 3.00 |
| 83 | 17 | 51 | 4.00 |

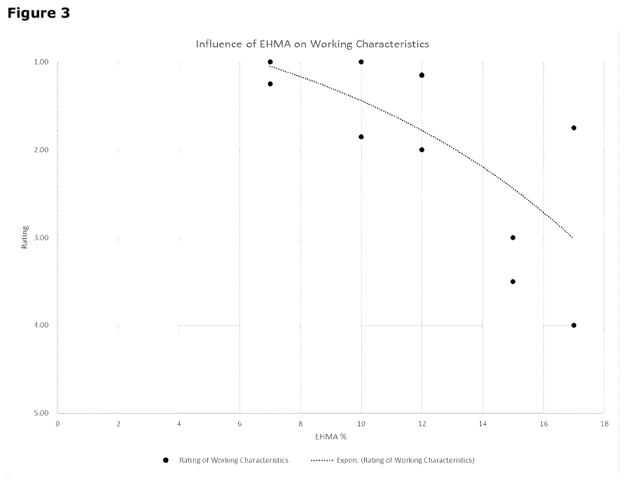

As shown in FIG. 3, charting the data in Table 14, working characteristics of the powder are observed to decline when EHMA content of the copolymer increases beyond 14%.

### Evaluation of Visual Defects

To assess visual defects in the artificial nail, a team of experienced nail industry professionals rated the individual combinations. Rating criteria for visual defects include the number of defects, the size of the defects and the visual clarity of the resulting enhancement. Samples were viewed with the naked eye and under microscope amplification, where the microscope equipment used was a Zeiss Axiolab 5 with 10× and 20× objectives. Known acrylic nail powders often have a Mv particle size near 60 microns. Testing copolymer ratios of this approximate particle size generated the following results shown in Table 15:

TABLE 15
Defects resulting from common particle size powder
Rating scale of 1-5, 1 being best/5 being worst

| EMA % in | EHMA % in | | Rating of Visual |
|---|---|---|---|
| Powder | Powder | Mv Particle Size | Defects |
| 83 | 17 | 51 | 3.75 |
| 85 | 15 | 51 | 2.25 |
| 88 | 12 | 58 | 2.00 |
| 90 | 10 | 63 | 2.25 |
| 93 | 7 | 54 | 4.25 |

Increasing the Mv particle size of the copolymer ratios illustrated some data points with reduced visual defects as shown in Table 16:

TABLE 16
Defects resulting from larger particle size powder
Rating scale of 1-5, 1 being best/5 being worst

| EMA % in | EHMA % in | | Rating of Visual |
|---|---|---|---|
| Powder | Powder | Mv Particle Size | Defects |
| 83 | 17 | 77 | 2.25 |
| 85 | 15 | 75 | 3.00 |
| 88 | 12 | 81 | 1.50 |
| 90 | 10 | 76 | 2.75 |
| 93 | 7 | 80 | 2.00 |

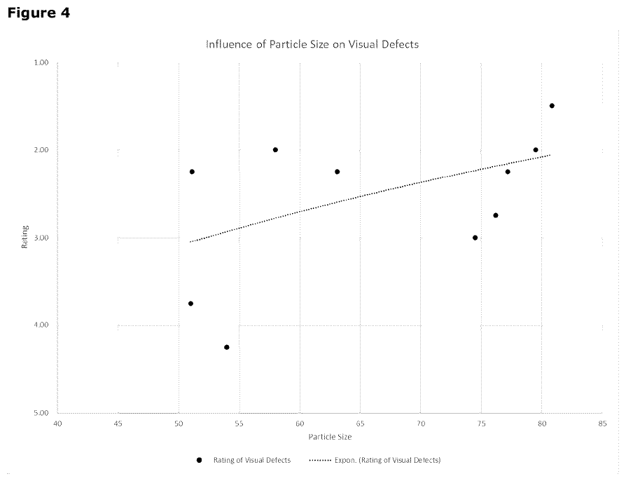

As shown in FIG. 4, by merging and charting the data in Table 15 and Table 16, within the composition of the present invention, visual defects decline when the Mv particle size of the powder increases.

Table 17 below uses the data in Table 15 and Table 16, rearranging the results to illustrate the relationship between the EMA/EHMA ratio and visual defects in the nail enhancement:

TABLE 17
Defects resulting from varying copolymer ratios
Rating scale of 1-5, 1 being best/5 being worst

| EMA % in | EHMA % in | | Rating of Visual |
|---|---|---|---|
| Powder | Powder | Mv Particle Size | Defects |
| 88 | 12 | 81 | 1.50 |
| 93 | 7 | 80 | 2.00 |
| 88 | 12 | 58 | 2.00 |
| 83 | 17 | 77 | 2.25 |
| 85 | 15 | 51 | 2.25 |
| 90 | 10 | 63 | 2.25 |
| 90 | 10 | 76 | 2.75 |
| 85 | 15 | 75 | 3.00 |
| 83 | 17 | 51 | 3.75 |
| 93 | 7 | 54 | 4.25 |

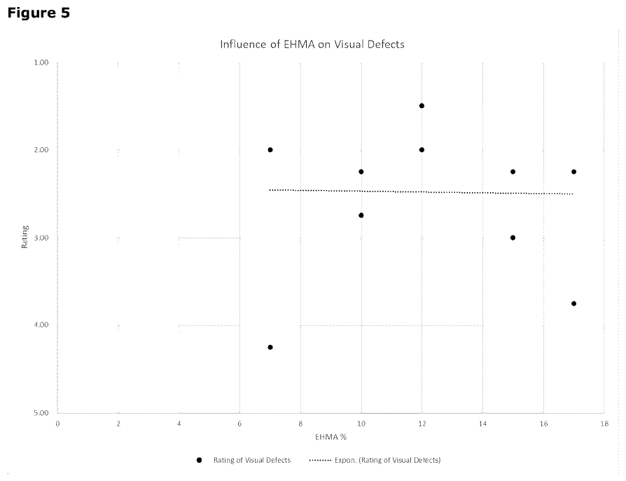

By charting the data in Table 17, an optimized combination of EMA/EHMA ratios was identified as shown in FIG. 5.

Using the above data analysis and as shown in Table 18, a preferred powder composition was identified for use with conventional (high EMA content) monomer.

TABLE 18
Combination of components showing desirable
working characteristics and performance

| Input | Best Results |
|---|---|
| Particle size influence on | Greater than 75 microns Mv |
| working characteristic | |
| EMA/EHMA ratio influence on | Between 88-94% EMA/6-12% EHMA |
| working characteristic | |
| Particle size influence on | Greater than 70 microns Mv |
| visual defects | |
| EMA/EHMA ratio influence on | 88% EMA:12% EHMA is preferred |
| visual defects | |

In other examples, preferred monomer liquid compositions may include one or more of di-, tri- and multi-functional (meth)acrylates such as glycerol di(meth)acrylate, triethylene glycol di(meth)acrylate and trimethylolpropane tri(meth)acrylate.

In other examples, preferred monomer liquid compositions may include one or more known amine accelerators, such as poly(oxy-1,2-ethanediyl), α,α′-[[(4-methylphenyl)imino]di-2,1-ethanediyl]bis[Ω-hydroxy- and or N-methyl-N-(2-hydroxypropyl)-ptoluidine and or N-ethyl-N-(2-hydroxyethyl)-p-toluidine.

In other examples, preferred monomer liquid compositions may include dyes and or colorants.

In other examples, the primary (meth)acrylate of preferred polymer powder compositions may comprise methyl (meth)acrylate and/or ethyl (meth)acrylate and/or n-butyl (meth)acrylate.

In other examples, the secondary (meth)acrylate of preferred polymer powder compositions may comprise 2-ethylhexyl (meth)acrylate and/or cyclohexyl (meth)acrylate and/or cetyl (meth)acrylate and/or hexyl (meth)acrylate and/or lauryl (meth)acrylate and/or stearyl (meth)acrylate.

In other examples, the preferred polymer powder compositions may comprise dibenzoyl peroxide and/or 2,4,6 (1H,3H,5H)pyrimidine trione.

In other examples, the preferred polymer powder contains a colorant.

In other examples, the preferred polymer powder contains a UV absorber, which may include benzotriazole (BTZ), hindered amine light stabilizers (HALS), triazine (HPT) UV absorbers and other free radical scavengers.

In other examples, the preferred polymer powder contains a polymerization inhibitor, which may include hydroquinone, 4-methoxyphenol, butylated hydroxytoluene and/or hydroxytoluene 4-tert-butylcatechol.

While the present invention has been described to include preferred embodiments, it is not intended that the scope of the invention be limited to the embodiments set forth herein, but instead to cover such alternatives, modifications, and equivalents as may be included within the spirit and scope of the invention as defined by the appended claims.

1. A nail-enhancing composition comprising:
a polymer component formed from:
a first (meth)acrylate ester with an alkyl side chain of 3 or less carbon atoms;
a second (meth)acrylate ester with a primary, secondary or tertiary alkyl side chain of at least 5 carbon atoms; and
an initiator.

2. A nail-enhancing composition comprising:
(i) a polymer component formed from:
a first (meth)acrylate ester with an alkyl side chain of 3 or less carbon atoms; and
a second (meth)acrylate ester with a primary, secondary or tertiary alkyl side chain of at least 5 carbon atoms; and an initiator; and
(ii) a monomer component comprising:
a first (meth)acrylate ester, where the (meth)acrylate ester is di-, tri-, or multi-functional;
a second (meth)acrylate ester with an alkyl side chain of 3 or less carbon atoms; and
an accelerator.

3. The nail-enhancing, low-odor composition of claim 2, wherein for the monomer component, the first (meth)acrylate ester is present in a greater amount by weight compared to the second (meth)acrylate ester.

4. The nail-enhancing composition of claim 2, wherein for the monomer component, the second (meth)acrylate ester is present in a greater amount by weight compared to the first (meth)acrylate ester.

5. (canceled)

6. (canceled)

7. The composition of claim 2 wherein the first (meth)acrylate ester of the monomer component is a di-, tri- or multi-functional (meth)acrylate ester.

8. The composition of claim 2 wherein the second (meth)acrylate ester of the monomer component has an alkyl side chain of 3 or less carbon atoms.

9. (canceled)

10. (canceled)

11. (canceled)

12. (canceled)

13. (canceled)

14. The composition of claim 2 wherein the first (meth)acrylate ester of the monomer component is selected from the group consisting of 1,3-butanediol di(meth)acrylate; 1,4-butanediol di(meth)acrylate; 1,6-hexanediol di(meth)acrylate; glycerol di(meth)acrylate; ethylene glycol di(meth)acrylate; diethylene glycol di(meth)acrylate; triethylene glycol di(meth)acrylate; tetraethylene glycol di(meth)acrylate; and combinations thereof.

15. (canceled)

16. (canceled)

17. The composition of claim 2 wherein the first (meth)acrylate ester of the monomer component is a blend selected from the group consisting of 1,3-butanediol di(meth)acrylate; 1,4-butanediol di(meth)acrylate; 1,6-hexanediol di(meth)acrylate; glycerol di(meth)acrylate; ethylene glycol di(meth)acrylate; diethylene glycol di(meth)acrylate; triethylene glycol di(meth)acrylate; tetraethylene glycol di(meth)acrylate; and the second (meth)acrylate ester is ethyl (meth)acrylate.

18. (canceled)

19. (canceled)

20. The composition of claim 2 wherein the monomer component has a theoretical vapor pressure of less than 10.0 mmHg at 20° C.

21. The composition of claim 2 wherein the ASTM E-679-04 dynamic dilution olfactometry odor concentration of the monomer liquid component is no more than 30,000 OU m³.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A method of forming an artificial nail enhancement, comprising:
applying the composition of claim 2 to a human nail;
forming from the applied composition a coating and/or artificial extension on a surface of the nail;
optionally extending the artificial nail beyond the length of the nail; and optionally buffing of the artificial nail to smooth its surface.

35. A method of forming an artificial nail enhancement, comprising:
wetting an applicator in a monomer liquid, the liquid comprising:
a first (meth)acrylate ester, where the (meth)acrylate ester is di-, tri or multi-functional;
a second (meth)acrylate ester with an alkyl side chain of 3 or less carbon atoms; and
an accelerator;
dipping the wetted applicator into a polymer powder for a time sufficient to allow the monomer on the applicator to mix with and absorb the polymer powder, where the polymer powder is formed from:
a first (meth)acrylate ester with an alkyl side chain of 3 or less carbon atoms;
a second (meth)acrylate ester with a primary, secondary or tertiary alkyl side chain of at least 5 carbon atoms; and
an initiator;
removing the applicator from the powder and allowing the resulting heterogeneous mixture present on the applicator to transition from two phases into one homogeneous mixture;
transferring the homogeneous mixture to a surface of a human nail; shaping the mixture with the applicator to form a coating and/or artificial nail extension;
optionally extending an artificial nail beyond the length of the nail; and optionally buffing the artificial nail to smooth its surface.

36. (canceled)

37. (canceled)

38. (canceled)

39. The method of claim 35, wherein the finished nail enhancement has an ASTM D-1003 transmission haze of no more than 40 candela.

40. (canceled)

41. An artificial nail kit containing
(i) a polymer component, where the polymer component is formed from:
a first (meth)acrylate ester with an alkyl side chain of 3 or less carbon atoms;
a second (meth)acrylate ester with a primary, secondary or tertiary alkyl side chain of at least 5 carbon atoms; and
a polymerization initiator;
(ii) a monomer component comprising:
a first (meth)acrylate ester, where the (meth)acrylate ester is di-, tri-, or multi-functional;
a second (meth)acrylate ester with an alkyl side chain of 3 or less carbon atoms; and
an accelerator; and
(iii) instructions for use.

42. (canceled)

43. (canceled)

44. (canceled)

45. (canceled)

46. (canceled)
